(19) United States
(12) Patent Application Publication
(10) Pub. No.: US 2024/0415890 A1
(43) Pub. Date: Dec. 19, 2024

(54) INFUSION OF FRESH IMMUNE EFFECTOR CELLS ARMED WITH MULTISPECIFIC ANTIBODY

(71) Applicant: University of Virginia Patent Foundation, Charlottesville, VA (US)

(72) Inventors: Lawrence G. Lum, Charlottesville, VA (US); Archana Thakur, Zion Crossroads, VA (US)

(73) Assignee: University of Virginia Patent Foundation, Charlottesville, VA (US)

(21) Appl. No.: 18/705,725

(22) PCT Filed: Oct. 31, 2022
(86) PCT No.: PCT/US2022/048491

§ 371 Date: Apr. 29, 2024

Related U.S. Application Data
(60) Provisional application No. 63/273,780, filed on Oct. 29, 2021.

(51) Int. Cl. A61K 35/17; A61K 39/00; A61P 35/00; C07K 16/28; C07K 16/32; C12N 5/0783; C12N 5/0787

(52) U.S. Cl. CPC A61K 35/17; A61K 39/4611; A61P 35/00; C07K 16/2809; C07K 16/2866; C07K 16/32; C12N 5/0636; C12N 5/0642; C07K 2317/31; C07K 2317/732; C12N 2501/2302

(57) ABSTRACT

A method for treatment of a patient suffering from a cancer, an autoimmune disease, an infection, a neurodegenerative disease or any combination thereof is disclosed. Compositions and uses are also disclosed. The method includes providing fresh immune effector cells (IECs) armed with one or more multispecific antibodies, wherein the multispecific antibodies are bound to the fresh IECs; and administering an effective amount of a composition of cells to the patient, wherein the composition of cells comprises the multispecific antibody bound fresh IECs. The fresh IECs cab comprise a cell selected from the group consisting of peripheral blood mononuclear cells (PBMC), antigen-presenting cells. T cells (optionally fresh Bi Ab armed T cells (FBATs)). NK cells, monocytes, polymorphonuclear neutrophils (PMNs), and any combination thereof. Providing the fresh IECs can comprise isolating fresh IECs from the patient. Arming fresh IECs can be accomplished with one or more multispecific antibodies, wherein the multispecific antibodies are bound to the fresh IECs.

### CROSS REFERENCE TO RELATED APPLICATIONS

This application claims priority to and benefit of U.S. Provisional Application Ser. No. 63/273,780 filed on Oct. 29, 2021, the disclosure of which is incorporated herein by reference in its entirety.

### TECHNICAL FIELD

The presently disclosed subject matter relates in some embodiments to methods for treating in any desired subject diseases and disorders, such as but not limited to diseases and disorders associated with cancer or autoimmune inflammation, such as but not limited to autoimmune diseases, graft-vs-host diseases, organ graft rejection, infections (viral, bacterial, fungal, and parasitic) and/or neurodegenerative disease (collectively AGOIN) with immune effector cells such as uncultured peripheral blood mononuclear cells (PBMC), peripheral blood T cells, T cell subsets, natural killer cells (NK cells), monocytes, and/or polymorphonuclear neutrophils (PMN) armed with multispecific antibodies (e.g., bispecific antibodies (BiAb)) directed at T cells, T cell subsets, NK cells, monocytes, and/or PMN and tumor associated target antigens or receptors on hematologic and solid malignancies and AGOIN target tissues.

### BACKGROUND

With the exception of B cell malignancies, current treatments for hematologic malignancies and solid tumors using CAR-T cells, tumor infiltrating lymphocytes (TILs), bispecific antibody (BiAb) alone, or ex vivo expanded activated T cells armed with BiAbs have had only very limited success.

It was previously believed that peripheral blood mononuclear cells (PBMCs), CAR-T cells, lymphokine activated killer cells (LAK) cells, purified T cells, or immune subsets must be activated and cultured prior to being armed with bispecific antibodies, including culturing for up to two (2) weeks.

Improved and novel therapeutic strategies are needed to treat cancers and other diseases and disorders, such as by cell reinfusion without ex vivo culture.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned: likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a method for treatment of a patient suffering from a cancer, an autoimmune disease, an infection, a neurodegenerative disease or any combination thereof, said method comprising the steps of: (a) providing fresh immune effector cells (IECs) armed with one or more multispecific antibodies, wherein the multispecific antibodies are bound to the fresh IECs; and (b) administering an effective amount of a composition of cells to the patient, wherein the composition of cells comprises the multispecific antibody bound fresh IECs.

In some embodiments, the fresh IECs comprises a cell selected from the group consisting of peripheral blood mononuclear cells (PBMC), antigen-presenting cells, T cells (optionally fresh BiAb armed T cells (FBATs)), NK cells, monocytes, polymorphonuclear neutrophils (PMNs), and any combination thereof.

In some embodiments, the method comprises providing the fresh IECs comprises isolating fresh IECs from the patient. In some embodiments, arming fresh IECs with one or more multispecific antibodies, wherein the multispecific antibodies are bound to the fresh IECs. In some embodiments, the IECs are autologous or allogenic to the patient. In some embodiments, the patient is given IL-2, IL-7, IL-12, IL-15, and/or GM-CSF and/or a checkpoint inhibitor. In some embodiments, the composition of cells comprises augmenting cytokines IL-2, IL-7, IL-15, IL-12, and/or GM-CSF and/or a checkpoint inhibitor.

In some embodiments, the method further comprises co-administering a composition of antigen-presenting cells (APC), and said FBATs armed with multispecific antibody: wherein the APC and the FBATs are autologous to the patient, and the patient is given IL-2, IL-7, IL-15, and/or GM-CSF and/or a check point inhibitor.

In some embodiments, the multispecific antibody comprises two monoclonal antibodies. In some embodiments, the multispecific antibody comprises a specificity for a tumor antigen. In some embodiments, the multispecific antibody comprises a specificity for a T cell receptor complex. In some embodiments, the multispecific antibody comprises specificities for a tumor antigen and a T cell receptor complex.

In some embodiments, wherein the multispecific antibody comprises monoclonal antibodies that are chemically heteroconjugated to form a bispecific antibody. In some embodiments, the multispecific antibody comprises an antibody construct of a plurality of specificities and a cytokine/chemokine receptor.

In some embodiments, the multispecific antibody comprises an antibody construct having at least three specificities wherein one of the at least three specificities comprises a T cell receptor complex. In some embodiments, the multispecific antibody comprises an antibody construct having at least three specificities and contains an antibody drug conjugate. In some embodiments, the multispecific antibody comprises an antibody construct having at least four specificities, wherein one the at least four specificities and contains a cytokine/chemokine receptor.

In some embodiments, an anti T cell receptor monoclonal antibody component of the multispecific antibody is directed against a CD3 of the T cell receptor complex.

In some embodiments, the patient is immunosuppressed. In some embodiments, the patient is susceptible to, or suffering from a disease associated with abnormal cellular proliferation or growth. In some embodiments, the patient is susceptible to, or suffering from a disease associated with an autoimmune reaction.

In some embodiments, the method further comprises freezing the composition of cells prior to administering the composition of cells to the patient in need of therapy. In some embodiments, the method further comprises thawing the composition of cells prior to administering the composition of cells to the patient in need of therapy.

In some embodiments, the IECs are armed with a dose of one or more multispecific antibodies of about 0.1 ng per million fresh IECs to about 1000 ng per million fresh IECs. In some embodiments, a dose administered to the patient is optimized for each individual patient by titrating fresh and thawed aliquot of the composition of cells to achieve a percent specific cytotoxicity level at an effector to target (E: T) ratio from about 10:1 to at least about 10% against a tumor target. In some embodiments, the dose administered to the patient is at least about 0.5×10⁶ the composition of cells per kilogram body weight of the patient with a dose schedule that can range from at least 1 administration to 24 administrations. In some embodiments, the dose administered to the patient is administered ranging from once a week to 1 once per month up to a year.

In some embodiments, the method further comprises further comprising infusing intravenously or injecting into a tumor arterial supply or tumor site said IECs armed with multispecific antibody: wherein said IECs armed with bispecific antibody are derived from an autologous donor or from an allogeneic donor.

In some embodiments, the composition of cells administered into a patient is free of soluble multispecific antibody.

In some embodiments, the cancer is selected from the group consisting of prostate cancer, breast cancer, leukemia, colon cancer, brain cancer, lung cancer, ovarian cancer, osteosarcoma, and neck cancer.

In some embodiments, the presently disclosed subject matter provides a method for treatment of a patient suffering from cancer, said method comprising the steps of: (a) isolating a sample of peripheral blood mononuclear cells, comprising T cells, from a patient suffering from cancer: (b) arming one or more of said T cells with multispecific antibody; (c) arming of said T cells with multispecific antibody capable of binding to the T cell receptor complex of a T cell, and to tumor-associated antigens on a tumor cell, under conditions wherein: (i) said bispecific antibody binds to said T cells, tumor cells, and Fc receptor positive cells, (ii) said antibody binds to the tumor target and said antibody binding to said tumor target activates said T cells, (iii) said antibody redirects said T cells and Fc-receptor positive cells to said tumor cells, (iv) said bispecific antibody activated T cells destroy said tumor cells; and, (d) reinfusing, into the patient, a composition of autologous cells comprising said bispecific antibody armed activated T cells (FBAT) as treatment of the patient wherein: (i) contacting T cells specific for different epitopes on the tumor cell with the autologous cells and including proliferation of the T cells specific for different epitopes on the tumor cells, or targeting and contacting multiple target cells with the armed T cell to which the BiAb remains bound, and killing said multiple target cells.

In some embodiments, the presently disclosed subject matter provides a method of treatment, comprising: (a) providing a formulation comprising: (i) a pharmaceutically acceptable excipient; and (ii) PBMC having bound thereto a bispecific antibody with a binding specificity for a T-cell antigen, selected from the group consisting of CD28 and CD3, and a binding specificity for a CD33, CD123, CD20, SLAMF7, BCMA, other liquid tumor antigens, HER2, EGFR, GD2, CEA, PSMA, PSA, VEGF, other solid tumor antigen present on a surface of a cancer cell, wherein the bispecific antibody has a binding specificity of about 10-8 moles/liter or higher: (b) infusing said formulation into a human patient suffering from cancer: (c) binding the bispecific antibodies to cancer cells in the patient: (d) contacting the cancer cells in the patient with the PBMCs and lysing the cancer cells in the patient: (e) creating a T cell memory in the patient's endogenous T cells; and (f) contacting cancer cells in the patient with the patient's endogenous T cells and lysing said cancer cells.

In some embodiments, the presently disclosed subject matter provides a method of preparing a composition of cells comprising (a) isolating fresh immune effector cells (IECs) from a patient; and (b) arming the fresh IECs with one or more multispecific antibodies, wherein the multispecific antibodies are bound to the fresh IECs without culture. In some embodiments, the method further comprises freezing the composition of cells. In some embodiments, the method further comprises (c) infusing into a subject armed IECs without culture or cryopreservation.

In some embodiments, the presently disclosed subject matter provides a composition produced by the foregoing methods.

In some embodiments, the presently disclosed subject matter provides a composition comprising fresh immune effector cells (IECs) armed with one or more multispecific antibodies, wherein the one or more multispecific antibodies are bound to the fresh IECs.

In some embodiments, the presently disclosed subject matter provides a composition for use in treating a patient suffering from a cancer, an autoimmune disease, a neurodegenerative disorder, an infection and/or any combination thereof, wherein the composition comprises fresh immune effector cells (IECs) armed with one or more multispecific antibodies, wherein the multispecific antibodies are bound to the fresh IECs.

In some embodiments, the presently disclosed subject matter provides use of a composition comprising fresh immune effector cells (IECs) armed with one or more multispecific antibodies, wherein the one or more multispecific antibodies are bound to the fresh IECs for the manufacture of a medicament for treating a cancer, an autoimmune disease, a neurodegenerative disorder, an infection and/or combination thereof. In some embodiments, the fresh IECs comprises a cell selected from the group consisting of peripheral blood mononuclear cells (PBMC), an antigen-presenting cells, T cell (optionally fresh BiAb armed T cells (FBATs)), NK cells, monocytes, polymorphonuclear neutrophils (PMNs), and any combination thereof.

In some embodiments of the composition or use, the fresh IECs are isolated from a patient, optionally a patient to be treated. In some embodiments of the composition or use, the IECs are autologous or allogenic to the patient. In some embodiments of the composition or use, the composition comprises augmenting cytokines IL-2, IL-7, IL-15, IL-12, and/or GM-CSF and/or a checkpoint inhibitor.

In some embodiments of the composition or use, the multispecific antibody comprises two monoclonal antibodies. In some embodiments of the composition or use, the multispecific antibody comprises a specificity for a tumor antigen. In some embodiments of the composition or use, the multispecific antibody comprises a specificity for a T cell receptor complex. In some embodiments of the composition or use, the multispecific antibody comprises specificities for a tumor antigen and a T cell receptor complex. In some embodiments of the composition or use, the multispecific antibody comprises monoclonal antibodies that are chemically heteroconjugated to form a bispecific antibody. In some embodiments of the composition or use, the multispecific antibody comprises an antibody construct of a plurality of specificities and a cytokine/chemokine receptor. In some embodiments of the composition or use, the multispecific antibody comprises an antibody construct having at least three specificities wherein one of the at least three specificities comprises a T cell receptor complex. In some embodiments of the composition or use, the multispecific antibody comprises an antibody construct having at least three specificities and contains an antibody drug conjugate. In some embodiments of the composition or use, the multispecific antibody comprises an antibody construct having at least four specificities, wherein one the at least four specificities and contains a cytokine/chemokine receptor.

In some embodiments of the composition or use, an anti T cell receptor monoclonal antibody component of the multispecific antibody is directed against a CD3 of the T cell receptor complex.

In some embodiments of the composition or use, the composition is frozen, optionally wherein the composition is thawed prior to administering the composition of cells to a patient in need of therapy.

In some embodiments of the composition or use, the IECs are armed with a dose of one or more multispecific antibodies of about 0.1 ng per million fresh IECs to about 1000 ng per million fresh IECs. In some embodiments of the composition or use, a dose administered to the patient is optimized for each individual patient by titrating fresh and thawed aliquot of the composition of cells to achieve a percent specific cytotoxicity level at an effector to target (E: T) ratio from about 10:1 to at least about 10% against a tumor target. In some embodiments, the dose administered to the patient is at least about 0.5×10⁶ the composition of cells per kilogram body weight of the patient with a dose schedule that can range from at least 1 administration to 24 administrations. In some embodiments, the dose administered to the patient is administered ranging from once a week to 1 once per month up to a year.

In some embodiments of the composition or use, the patient is immunosuppressed. In some embodiments of the composition or use, the patient is susceptible to, or suffering from a disease associated with abnormal cellular proliferation or growth. In some embodiments of the composition or use, the patient is susceptible to, or suffering from a disease associated with an autoimmune reaction.

In some embodiments of the method, composition or use, the composition further comprises a pharmaceutically acceptable carrier, excipient, and/or diluent, optionally wherein the pharmaceutically acceptable carrier, excipient, and/or diluent is pharmaceutically acceptable for use in a human.

Accordingly, it is an object of the presently disclosed subject matter to provide compositions and methods for treating diseases or disorders characterized by cancers, autoimmune, inflammatory, and degenerative conditions. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A and 1B. FIG. 1A showing SDS-PAGE images of heteroconjugated products of CD33GOBi and CD123Bi. Lane 1 in both gels: molecular weight markers (MW): Lane 2 in both gels: OKT3: Lane 3: CD33 in gel #1 or CD123 in gel #2: Lane 4; CD33GOBi in gel #1 or CD123Bi in gel #2 containing unconjugated monomers, dimers (arrow), and multimers forms. FIG. 1B shows the relative expression of CD33 and CD123 on various AML cell lines.

FIGS. 2A and 2B. Arming Dose Titration of BiAb. Normal donor ATC (n=3) were left unarmed (UA, 0) ng/106 ATC) or armed at 3, 6, 12, 25, 50 and 10⁰ ng/106 ATC with CD123Bi (FIG. 2A) and CD33GOBi (FIG. 2B) and tested against KG1 and EOL1 at E: T 1:1 in an 18 hr. cytotoxicity assay. The difference between armed and unarmed ATC was highly significant (p<0.0001) for 3, 6, 12, 25, 50 and 100 ng/106 ATC for both cell lines.

FIGS. 3A to 3F: E: T titration at BiAb Arming Dose of 25 ng/106 ATC (n=3). Cytotoxicity of CD33GO- or CD123 BATs against TF1 (FIG. 3A), KG1 (FIG. 3B), EOL1 (FIG. 3C), NoMo1 (FIG. 3D), HL60 (FIG. 3E) and HL60/VCR (FIG. 3F) cell lines was measured at fixed arming dose of 25 ng/10⁶ ATC at 0.5:1, 1:1, 2:1 and 4:1 E/T ratios. Unarmed ATC (UA) or ATC armed at 25 ng/10⁶ cells with CD33GOBi, CD123Bi or combining both BATs CD33GO−/CD123 BATs at ET ratio of 1:1 were incubated for 18 hr. with AML cell lines listed above showed increasing cytotoxicity from 0.5:1 to 2:1 ratio plateauing at E: T of 4:1 for KG1, EOL1, HL60 and HL60/VCR cell lines with the exception of NoMo1, TF1 which showed gradual increase at E: T from 0.5:1 to 4:1.

FIG. 4. Treatment Schema with AML engrafted in NSC Mice. In vivo Efficacy of CD33GO and CD123 BATs was tested against AML in NSG Mice. OD scid gamma ([NSG] NOD.Cg-Prkdc^{scid} 112rg^{tmIWjI}//SzJ) mice 6-8-week-old were irradiated with 250 Rads, after 24 h of irradiation mice were intravenously injected with 10×10⁶ KG1 cells to establish human AML graft.

FIGS. 5A to 5B. FIG. 5A shows the average weight of all four groups from days 21-52 and FIG. 5B shows the survival proportions of all four groups (n=8 mice/group). Mice treated with BATs received 20×10⁶cells/injection, 3x/week for 4 weeks and mice treated with GO (My lotarg) received 0.06 mg/kg GO alone 3x/week for 4 weeks.

FIGS. 6A to 6D. Induction of FBATs directed at CD33 and CD123 by arming fresh PBMC with anti-CD3 x anti-CD33 or anti-CD3 x anti-CD123. FIG. 6A panel summarizes killing by Fresh PBMC alone, GO alone, CD33GOBi alone, CD123Bi alone or PBMC+GO, PBMC+CD33GOBi, PBMC+CD123GOBi. Cytotoxicity was significantly higher (P<0.0001) by PBMC+CD33GOBi and PBMC+CD123GOBi compared to PBMC alone, GO alone, CD33GOBi alone, CD123Bi alone or PBMC+GO at E/T 2:1 against EOL1 cell line. FIG. 6B, FIG. 6C, and FIG. 6D panels shows the results for fresh PBMC for the 3 different normal subjects (VA02, VA07, and VA13).

FIGS. 7A to 7C. FIG. 7A shows cytotoxicity of rEGFR BATs armed at 0.1-200 ng BiAb cEGFRBi/106 cells vs. cEGFRBi BATs and HER2Bi BATs armed at 50 ng BiAb/106 cells. For MCF-7 cells, there was no significant differences among any of the rEGFR arming concentrations except for 0.1 ng arming dose and those armed with 50 ng of cEGFRBi (▪) or HER2Bi (▴). For MDA-MB-231 (FIG. 7B) and MiaPaCa-2 cells (FIG. 7C), 25-200 arming doses of rEGFRBi showed 75-100% cytotoxicity. Arming concentrations of 0.1 and 1 ng did not show any cytotoxicity nor did the cEGFR BATs or HER2 BATs armed at 50 ng/106 cells for both cell lines.

FIGS. 8A to 8B. Shows cytotoxicity by BATs incubated with solid tumor cell lines, rEGFR BATs armed at 50 ng dose from three healthy donors showed significantly higher cytotoxicity at 1:1 ET ratio compared to HER2 BATs and cEGFR BATs against MCF-7 (FIG. 8A) and MDA-MB-231 (FIG. 8B) cells in RTCA system. * P<0.05.

FIG. 9. Fresh PBMC armed with rEGFRBi maintain cytotoxicity over a broad range of arming concentrations. Similar to arming ATC, fresh PBMC were armed with rEGFRBi at arming doses ranging from 0.1-200 ng/10° PBMC. Cytotoxic activity by armed PBMC showed increasing cytotoxicity with time against MCF-7 cells, cytotoxicity at arming doses of 1-100 ng ranged between 50-75% at 96 h. The same pattern was seen with a normal donor armed PBMC against MDA-MB-231, MiaPaCa-2 cells.

FIG. 10A to 10D. Shows the cytotoxicity by PBMC against MCF-7 cell line compared to PBMC stimulated with OKT3. Cytotoxicity of rEGFRBi (20 ng/106) PBMC stimulated PBMCs at days 2 (FIG. 10A) (at 12 and 24 h time point), 3 (FIG. 10B), 6 (FIG. 10C), and 9 (FIG. 10D) (at 24, 48 and 72 h time point) were significantly higher compared to cEGFR (20 ng/106) PBMC or OKT3 (20 ng/106 PBMC) stimulated T cells at 1:1 E/T ratio in xCELLigence RTCA system.

FIG. 11. Shows the cytotoxicity by fresh armed PBMC and 14-day cultured ATC armed with 50 ng/106 rEGFRBi (BATs) against MCF-7 cell line. Cytotoxicity mediated by rEGFRBi armed ATC (BATs) was determined at various E/T ratios ranging from 5:1-1:5 at arming dose of 50 ng/106 ATC in xCELLigence RTCA system Cytotoxicity of rEGFRBi (50 ng/106) PBMC stimulated PBMC was determine at various E: T ranging from 5:1-1:5 at 24, 48, 72, 96, and 120 hours. Unarmed 14-day ATC and unarmed PBMC are the controls.

FIG. 12A to 12B. Shows the proliferation of freshly armed PBMC armed with 50 ng/106 rEGFRBi (FIG. 12A, linear fold) or chemically heterconjugated anti-CD3 x anti-EGFR (cEGFRBi) without engagement of tumor targets (FIG. 12B, log fold of the same data). The fold increases induced by rEGFRBi and cEGFRBi were compared to fold increases induced by OKT3 (20 ng/ml).

FIG. 13. Induction of Cytotoxicity Directed at MCF-7 by armed PBMC with Her2Bi and cEGFRBi. Chemical BiAbs Induce FBATs. The ability of chemical heterconjugated cHER2Bi and cEGFRBi, and rEGFRBi to induce cytotoxicity was tested over time in the RTCA system at a low E: T of 1:1, against MCF-7 cell line.

FIG. 14. Shows experiments performed in which fresh PMNs armed with anti-CD89 x anti-HER2 bispecific antibody enhanced neutrophil mediated killing of Sk-BR-3 breast cancer cell line at E: T ratios from 10:1 to 20:1.

FIG. 15. Shows Fresh PBMC were activated at various doses (0.5, 5.0, 50 and 500 ng/106 FPBMC) of SLAMF7Bi compared standard 50 ng-OKT3 induced activation, following activation armed FPBMC were tested for their cytotoxic activity at 0, 48 and 144 hours of culture against MM. IS multiple myeloma cell line

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein: rather, these embodiments are provided so that this disclosure will satisfy applicable legal requirements.

### I. Definitions

While the following terms are believed to be well-understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including.” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause: other elements are not excluded from the claim as a whole.

As used herein, the phrase “consisting essentially of”limits the scope of a claim to the specified materials or steps, plus those that do not materially affect the basic and novel characteristic(s) of the claimed subject matter.

Unless otherwise indicated, all numbers expressing quantities of time, temperature, light output, atomic (at) or mole (mol) percentage (%), and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

With respect to the terms “comprising,” “consisting essentially of,” and “consisting of,” where one of these three terms is used herein, the presently disclosed and claimed subject matter encompasses the use of either of the other two terms. For example, “comprising” is a transitional term that is broader than both “consisting essentially of” and “consisting of,” and thus the term “comprising” implicitly encompasses both “consisting essentially of” and “consisting of.” Likewise, the transitional phrase “consisting essentially of” is broader than “consisting of,” and thus the phrase “consisting essentially of” implicitly encompasses “consisting of.”

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, a subject is a human. The terms “subject” and “patient” are used interchangeably herein.

Similarly, all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos, for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of cells from livestock, including but not limited to domesticated swine (pigs and hogs), ruminants (cattle), horses, poultry, and the like. Thus, provided in accordance with presently disclosed subject matter are non-human animal or veterinary treatments of any disease or disorder corresponding to a human treatment as described herein. Representative such diseases include diseases and disorders associated with cancer, inflammation (such as autoimmune inflammation), autoimmune diseases, graft-vs-host diseases, organ graft rejection, infections (viral, bacterial, fungal, and parasitic) and/or neurodegenerative disease (collectively AGOIN).

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent.” as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, cancer. The additional compounds can also be used to treat symptoms associated with the injury, disease, or disorder, including, but not limited to, pain and inflammation.

The term “adult” as used herein, is meant to refer to any non-embryonic or non-juvenile subject.

A disease or disorder is “alleviated” if the severity of a symptom of the disease, condition, or disorder, or the frequency with which such a symptom is experienced by a subject, or both, are reduced.

“Allogeneic” refers to cells or to a graft derived from a different individuals of the same species.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | Functionally Equivalent |
|---|---|---|---|
| Full Name | Code | Code | Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an amino acid in which the side chain is fused to the amino group.

Amino acids have the following general structure:

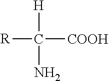

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “fresh,” or “freshly” as used herein, refers to the harvested cell population that is uncultured.

The term “freshly armed,” as used herein, refers to a cell or cells that have been armed as described herein with a multispecific antibody or other reagent. Furthermore, freshly arming includes not culturing the cells to be armed with a multispecific antibody or other reagent. These immune cells can be “freshly armed” and cryopreserved in multiple aliquots to be thawed and infused at multiple time points during therapy.

The term “immune effector cell” or “immune effector cells,” (IEC) as used herein, refers to a cell or cells from a subject that are capable of modulating or effecting an immune response. IECs include, but are not limited to, peripheral blood mononuclear cells (PBMC), T cells (fresh BiAb armed T cells (FBATs), NK cells, monocytes, polymorphonuclear neutrophils (PMNs) and any combination thereof.

The term “fresh immune effector cell,” “fresh IEC”, “IECs”, and “IEC” are used herein interchangeably in some aspects.

The term “armed immune effector cell,” “fresh armed immune effector cell” “armed IECs” “IECs armed with a multispecific antibody” and “composition of cells” are used herein interchangeably in some aspects. In some embodiments, the fresh armed IECs comprise minimally manipulated fresh cells. In some embodiments, the minimally manipulated fresh cells are armed with multispecific antibodies or other reagents and not cultured.

The term “antibody,” as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F (ab) 2 and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F (ab) 2 a dimer of Fab which itself is a light chain joined to V_{H}-CHI by a disulfide bond. The F (ab) 2 can be reduced under mild conditions to break the disulfide linkage in the hinge region, thereby converting the F (ab) 2 dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain,” as used herein, refers to the larger of the two types of polypeptide chains present in all intact antibody molecules.

An “antibody light chain,” as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567:5, 482,856:6,479,284:6,677,436; 7,060,808:7,906,625:8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “multispecific antibody” or “multispecific antibodies” as used herein, refers to an antibody that combines two or more antigen-recognizing elements into a single antibody molecule that is able to bind to two or more targets. This includes bispecific antibodies and trispecific antibodies or other antibody constructs capable of binding to a receptor complex of an immune effector cell, such as a T cell receptor complex of a T cell, and to an antigen on a cell, such as a tumor-associated antigen on a tumor cell. Multispecific antibodies are bioengineered to contain multiple distinct antigen-binding domains or specificities, such as but not limited to two, three, four or more distinct antigen-binding domains or specificities. In some embodiments, the multispecific antibody is a multivalent antibody and refers to an antibody that binds to multiple sites on one target.

The term “arm” or “arming” as used herein, refers to attaching multispecific antibodies that recognize molecules or antigens on molecules on cancer or inflammatory cells to immune effector cells that are effective at eliminating cancer or inflammatory cells. In some embodiments, the multispecific antibody or other reagent is attached to the immune effector cell by chemical heteroconjugation.

The term “antigen” as used herein is defined as a molecule that provokes an immune response. This immune response can involve either antibody production, or the activation of specific IECs, or both. By way of example and not limitation, an antigen can be derived from organisms, subunits of proteins/antigens, killed or inactivated whole cells or lysates.

The term “autologous,” as used herein, refers to something (e.g., a cell or cells) that occurs naturally and normally in a certain type of tissue or in a specific structure of the body. In transplantation, it refers to a cell transplant or a graft in which the donor and recipient are the same individual, or to blood that the donor has previously donated and then receives back, usually during surgery.

The term “allogenic,” as used herein, refers to something (e.g., a cell or cells) which are taken from one or more individuals and given to a different individual of the same species.

The term “basal medium,” as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “biocompatible,” as used herein, refers to a material that does not elicit a substantial detrimental response in the host.

The term “biodegradable,” as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample,” as used herein, refers to samples obtained from a living organism, including skin, hair, tissue, blood, plasma, cells, sweat, and urine.

The term “bioresorbable,” as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable,” as the term is used herein, means that at least 80% of the total mass of material implanted is resorbed within one year.

A “bispecific antibody,” as used herein, refers to an antibody having binding specificities for at least two different antigenic epitopes. In some embodiments, the epitopes are from the same antigen. In some embodiments, the epitopes are from two different antigens. Methods for making bispecific antibodies are known in the art. For example, bispecific antibodies can be produced using recombinant technology using the co-expression of two immunoglobulin heavy chain/light chain pairs. See, e.g., Milstein et al. (1983) Nature 305:537-39. Alternatively, bispecific antibodies can be prepared using chemical linkage. See, e.g., Brennan et al. (1985) Science 229:81. Bispecific antibodies include bispecific antibody fragments. See, e.g., Bolliger et al. (1993) Proc. Natl. Acad. Sci. U.S.A. 90:6444-48, Gruber et al. (1994) J. Immunol. 152:5368.

Any suitable or desired bispecific antibody as would be apparent to one of ordinary skill in the art upon a review of the instant disclosure can be employed. Techniques and examples of bispecific antibodies are disclosed in the art. See, for example, U.S. Pat. No. 7,763,243, U.S. Patent Application Publication No. 2018/0282693, U.S. Patent Application Publication No. 2018/0243341, and U.S. Patent Application Publication No. 2019/0343954, each of which is hereby incorporated by reference in its entirety. Additional examples are also described, such as in Thakur et al., Oncoimmunology. 2018 Aug. 27: 7 (12): Vaishampayan et al., Prostate Cancer. 2015:2015:285193, Epub 2015 Feb. 23: Lum et al., Clin Cancer Res. 2015 May 15: 21 (10): 2305-14, Epub 2015 Feb. 16; Zitron et al., BMC Cancer. 2013 Feb. 22:13:83; Yankelevich et al., Pediatr Blood Cancer. 2012 Dec. 15: 59 (7): 1198-205: Zhao et al., J Appl Physiol 104:1793-1800, 2008: Sen et al., J Hematother Stem Cell Res 2001 10:247-60; Lum et al., Clinical Breast Cancer, Vol. 4, No. 3, 212-217, 2003: Lum, Expert Opin. Drug Discov. (2008) 3 (9): 1-17: Lum et al., Biol Blood Marrow Transplant 18:1012-1022 (2012): Lum et al., Biol Blood Marrow Transplant 19 (2013) 925-933. In some embodiments, the bispecific antibody (BiAb) is a chemically heteroconjugated bispecific antibody or a recombinant bispecific antibody of any configuration.

Any suitable bispecific antibody and technique for the production thereof as would be apparent to one of ordinary skill in the art upon a review of the instant disclosure falls within the scope of the presently disclosed subject matter. In some embodiments, the presently disclosed subject matter employs bispecific antibodies (BiAbs) produced by chemical joining of two monoclonal antibodies. Examples of bispecific antibodies and techniques for producing bispecific antibodies are known the art and have been described in several reviews, along with their respective target antigens and T cell antigens. Representative reviews include Thakur, A., and Lum, L. G.: Cancer therapy with bispecific antibodies: Clinical experience. Current Opinion and Molecular Therapeutics 12:340-349, 2010; Lum, L. G., and Thakur, A.: Bispecific Antibodies for Arming Activated T Cells and Other Effector Cells for Tumor Therapy. Book Chapter in: Bispecific Antibodies. Kontermann, R. E. (ed). Germany: Springer Heidelberg, 2011, pp. 243-271; Lum, L. G., and Thakur, A.: Targeting T Cells with Bispecific Antibodies for Cancer Therapy: A Review. BioDrugs 25:365-379, 2011; and Thakur, A., Huang, M., Lum, L. G.: Bispecific antibody based therapeutics: Strengths and challenges. Blood Reviews, 2018 (Impact 6.6). Representative U.S. patents relating to BiAbs and production thereof include U.S. Pat. No. 10,550,193:10,519,247:10,294,300; 10,239,951; and 10,179,819, each of which is herein incorporated by reference in its entirety.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, |
| | polar residues | Gly |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A tissue “normally comprises” a cell if one or more of the cells are present in the tissue in an animal not afflicted with a disease or disorder.

A “compound,” as used herein, refers to any type of substance or agent that is commonly considered a drug, or a candidate for use as a drug, combinations, and mixtures of the above, as well as polypeptides and antibodies of the presently disclosed subject matter.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine,” as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as one or more small chemical groups on the antigen molecule that can elicit and react with an antibody. An antigen can have one or more epitopes. Most antigens have many epitopes: i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci USA 87:2264-2268, modified as in Karlin & Altschul (1993) Applications and statistics for multiple high-scoring segments in molecular sequences. Proc Natl Acad Sci USA 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci USA 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5: gap extension penalty=2: mismatch penalty=3: match reward =1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI-BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G: C ratio within the nucleic acids.

The term “ingredient” refers to any compound, whether of chemical or biological origin, that can be used in cell culture media to maintain or promote the proliferation, survival, or differentiation of cells. The terms “component,” “nutrient,” “supplement,” and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit,” as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block.”

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “administering,” “infusing,” “reinfusing.” “transplanting.” “injecting” or “applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal approach. As used herein, “injury” generally refers to damage, harm, or hurt: usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select.”

The terms “isolate,” “isolated,” “isolating,” and grammatical variations thereof when used in reference to compositions or cells, refers to a single composition or cell of interest, or a population of compositions or cells of interest, at least partially isolated from other cell types or other cellular material with which it occurs in a culture or a tissue of origin.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate,” as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e. analogs having other than a phosphodiester backbone. For example, the so called “peptide nucleic acids,” which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphorami date, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end: the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand:” sequences on the DNA strand which are located 5′ to a reference point on the DNA are referred to as “upstream sequences”: sequences on the DNA strand which are 3′ to a reference point on the DNA are referred to as “downstream sequences”.

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of cells, graft or a pharmaceutical composition includes any route of administration or infusion characterized by physical breaching of a tissue of a subject and administration of the pharmaceutical composition through the breach in the tissue. Parenteral administration thus includes, but is not limited to, administration of cells, graft or a pharmaceutical composition by injection, by application through a surgical incision, by application through a tissue-penetrating non-surgical wound, and the like. In particular, parenteral administration is contemplated to include, but is not limited to, topical, oral, intravenous, intramuscular, intra-arterial, intrasternal, intratumoral, intramedullary, intrathecal, intraventricular, transdermal, subcutaneous, intraperitoneal, intranasal, enteral, kidney dialytic infusion, sublingual, or rectal approaches.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer.”

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl: aromatic urethane protecting groups such as benzyloxy carbonyl; and aliphatic urethane protecting groups, for example, tert-butoxy carbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus: the right-hand end of a polypeptide sequence is the carboxyl-terminus.

The term “protein regulatory pathway,” as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.” A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample,” as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support,” “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds,” as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof”is a patient, e.g., animal, mammal, or human, who will benefit from a method, a use or a composition of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence: e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2X standard saline citrate (SSC), 0.1% SDS at 50° C.: in some embodiments in 7% (SDS), 0.5 M NaPO4, 1 mM EDTA at 50° C. with washing in 1×SSC, 0.1% SDS at 50° C.: in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a: Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function: (2) a part of an organism consisting of an aggregate of cells having a similar structure and function: or (3) a grouping of cells that are similarly characterized by their structure and function, such as muscle or nerve tissue.

The term “topical application,” as used herein, refers to administration to a surface, such as the skin. This term is used interchangeably with “cutaneous application” in the case of skin. A “topical application” is a “direct application.”

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous.”

The term “transfection” is used interchangeably with the terms “gene transfer,” “transformation,” and “transduction,” and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polyline compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cossids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

In some embodiments, antibodies are single chain, monoclonal, multispecific, bispecific, trispecific, synthetic, polyclonal, chimeric, human, or humanized, or active fragments or homologs thereof. In some embodiments, the antibody binding fragment is scFV, F (ab) 2, F (ab) 2, Fab′, or Fab. Fragments within the scope of the term “antibody.” include those produced by digestion with various proteases, those produced by chemical cleavage and/or chemical dissociation and those produced recombinantly, so long as the fragment remains capable of specific binding to a target molecule. Among such fragments are Fab, Fab, Fv, F (ab) 2, and single chain Fv (scFv) fragments. In some embodiments, the specific binding molecule is a single-chain variable (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bispecific scFvs are sometimes referred to as diabodies. Fragments within the scope of the term “antibody” include those produced by digestion with various proteases, those produced by chemical cleavage and/or chemical dissociation and those produced recombinantly, so long as the fragment remains capable of specific binding to a target molecule (i.e., comprise at least one paratope).

Other representative patent documents disclosing techniques relating to antibody production include the following, all of which are herein incorporated by reference in their entireties: PCT International Patent Application Publication Nos. WO 1992/02190 and WO 1993/16185: U.S. Patent Application Publication Nos. 2004/0253645, 2003/0153043, 2006/0073137, 2002/0034765, and 2003/0022244; and U.S. Pat. Nos. 4,816,567:4, 946,778:4,975,369:5,001,065:5,075,431:5,081,235:5,169,939; 5,202,238; 5,204,244:5, 225,539:5,231,026; 5,292,867:5,354,847; 5,436,157:5,472,693:5,482,856:5,491,088:5, 500,362:5,502,167:5,530,101:5,571,894; 5,585,089:5,587,458:5,641,870:5,643,759:5, 693,761:5,693,762:5,712,120:5,714,350:5,766,886; 5,770,196; 5,777,085:5,821,123: 5,821,337:5,869,619:5,877,293:5,886,152:5,895,205:5,929,212:6,054,297:6,180,370; 6,407,213:6,548,640:6,632,927:6,639,055:6,750,325; and 6,797,492. The presently disclosed subject matter is also directed to methods of administering the compositions of the presently disclosed subject matter to a subject.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

The presently disclosed subject matter encompasses the preparation and use of pharmaceutical compositions for treatment of the diseases and disorders disclosed herein. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Patients to which administration of the pharmaceutical compositions of the presently disclosed subject matter include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient. It can generally be stated that a pharmaceutical composition comprising the cells described herein may be administered at dosages described herein. Compositions may also be administered multiple times at these dosages. The cells can be administered, for example, by using infusion techniques that are commonly known in immunotherapy (see, e.g., Rosenberg et al., New Eng. J. of Med. 319:1676, 1988). Upon a review of the instant disclosure, dosage and treatment regime for a particular subject can be determined by one skilled in the art of medicine by monitoring the subject for signs of disease and adjusting the treatment accordingly.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents: dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pennsylvania, United States of America and/or Gennaro (ed.) (2003) Remington: The Science and Practice of Pharmacy, 20th edition Lippincott, Williams & Wilkins, Philadelphia, Pennsylvania, United States of America, each of which is incorporated herein by reference.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the compositions of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

### II. Representative Embodiments

In some embodiments, the presently disclosed subject matter arms fresh or uncultured PBMC, T cells or T cell subsets, TILs, LAK, polymorphonuclear neutrophils (PMN) with BiAbs for reinfusion without ex vivo culture.

In some embodiments, the presently disclosed subject matter provides bispecific antibody activated T cells (also referred to herein as BAT cells) in which a subject or patient's T-cells are harvested and armed with bispecific antibody ex vivo, and added back to the patient. However, any desired cell of the immune system can be harvested, armed, and added back to the patient. Representative cells include but are not limited to PBMCs, peripheral blood T cells, T cell subsets and other immune cell subpopulations, natural killer cells (NK cells), monocytes, buffy coat cells, and/or polymorphonuclear neutrophils (PMN). In some embodiments, the presently disclosed subject matter employs PBMCs, instead of purified T-cells.

In some embodiments, the presently disclosed subject matter involves only treating cells (such as but not limited to peripheral blood mononuclear cells (PBMC), peripheral blood T cells. T cell subsets, natural killer cells (NK cells), monocytes, buffy coat cells, and/or polymorphonuclear neutrophils (PMN) armed with bispecific antibodies (BiAb) directed at T cells, T cell subsets and other immune cell subpopulations, NK cells, monocytes, and/or PMN) for a few hours and/or days, or less. In some embodiments, the cells can be used immediately after harvesting and washing via apheresis. In some embodiments, the cells might be held in a suitable medium for up to 1, 12, 24, 48, and/or 72 hours. In some embodiments, the cells might be held in a suitable medium for up to 1 hour. In some embodiments, the cells might be held in a suitable medium for up to 12 hours. In some embodiments, the cells might be held in a suitable medium for up to 24 hours. In some embodiments, the cells might be held in a suitable medium for up to 48 hours. In some embodiments, the cells might be held in a suitable medium for up to 72 hours.

In some embodiments, such a medium can be free of any reagent typically used in culturing BAT. Such cells can be called uncultured, as opposed to the 2-week culture that has been historically used for BATs. In some embodiments, the use of unmanipulated, uncultured cell populations forms an aspect of the presently disclosed subject matter.

In some embodiments, the method can be applied to hematologic malignancies and solid tumors that are included but are not a fully inclusive list of cancers to be treated by PBMC. T cells or T cell subsets, or other effectors armed with BiAbs.

Acute myeloid leukemia. Acute myeloid leukemia (AML) is a heterogeneous disease characterized by the accumulation of malignant cells in the myeloid lineage that pose a significant therapeutic challenge¹. Despite of advances in the understanding of AML biology and genetics, the chemotherapy used to treat AML has not significantly changed in four decades²⁻⁷. Although allogeneic stem cell transplant (AlloSCT) provides long term cure for high risk patients who have an AlloSCT donor, there are significant regimen related toxicities and a high risk of relapse⁸⁻¹⁴. Recurrent disease is attributed to leukemic stem cells (LSCs), which are thought to be resistant to chemotherapy and capable of reinitiating the disease. Since leukemic antigens CD33 and CD123 are highly expressed on blasts and LSCs with very low or undetectable expression on hematopoietic stem cells, make them attractive AML targets^{15,16}. Although a humanized anti-CD33 IgG4 antibody conjugated to the cytotoxic agent calicheamicin (gemtuzumab ozogamicin, [GO]) was reapproved for treating newly diagnosed AML in relapsed/refractory (R/R) AML in adults and pediatric patients, treatment with GO continues to be associated with serious adverse effects^{17,18}

In some embodiments, the presently disclosed subject matter can be used, for example, to treat AML by arming fresh PBMC, T cell or T cell subsets from AML patients in early relapse or PBMC from the HLA-identical or haploidentical donor with anti-CD3 x anti-CD33 BiAb.

Non-Hodgkin's Lymphoma. High-dose chemotherapy (HDC) followed by autologous stem cell transplantation (SCT) induces complete responses in patients with high-risk, refractory, or relapsed non-Hodgkin's lymphomas (NHLs).¹⁹⁻²² In the rituximab era where rituximab added to cyclophosphamide, doxorubicin, vincristine, and prednisone (R-CHOP) improves progression free survival over CHOP,²³ only 10% of chemosensitive patients who do not achieve a complete response or who are in relapse remain disease free with salvage chemotherapy alone.²⁴

In some embodiments, NHL can be treated with fresh autologous PBMC armed with anti-CD3 x anti-CD20 BiAb as part of consolidation or after autologous SCT to further decrease the amount of lymphoma.

Acute Lymphocytic Leukemia. CAR-T targeting CD20 and CD19 have been approved for Acute Lymphocytic Leukemia (ALL) and blinatumumab (anti-CD3 x anti-CD19 BiAb) has been approved for ALL. Life-threatening cytokine release syndrome (CRS) or cytokine storm continues to make management of the patients difficult. In accordance with the presently disclosed subject matter, fresh PBMC obtained from the patient (donor-derived T cells) or fresh donor PBMC are armed with anti-CD3 x anti-CD20 or anti-CD3 x anti-CD19 BiAb for the treatment of relapse after allogeneic SCT.

In some embodiments, fresh PBMC are armed with anti-CD3 x anti-CD20 or anti-CD3 x anti-CD19 BiAbs in combination with chemotherapy for the treatment of primary ALL.

Multiple Myeloma. Multiple myeloma (MM) is the second most common hematologic malignancy. Patients are the most sensitive and responsive to the first line of therapy, which provides the highest chance of achieving minimal residual disease (MRD) negativity. With subsequent lines of therapy, the depth and duration of response typically lessens and many patients ultimately become refractory to treatment. With the introduction of proteasome inhibitors (PIs), immunomodulatory agents (IMiDs), histone deacetylase (HDAC) inhibitors and monoclonal antibodies (mAb), the number of patients achieving 5 year survival in 2019 was over 50%. Despite the effectiveness of combination therapies, autologous stem cell transplant (autoSCT) and maintenance, MM remains an incurable disease.

In some embodiments, the presently disclosed subject matter can be used to treat patients who have failed 3 drugs by arming fresh PBMC with anti-CD3 x anti-CS-1 or anti-CD3 x anti-BCMA BiAb to treat MM or arming fresh PBMC with anti-CD3 x anti-CS-1 or anti-CD3 x anti-BCMA BiAb to treat MM after autologous SCT as consolidation therapy or treatment after relapse of MM after autologous SCT.

Prostate Cancer. Prostate cancer (PC) is the most common cancer in American men. Most of the deaths related to PC are from metastatic disease. Despite recent advances in treatment options, progression eventually occurs and metastatic castrate resistant prostate cancer (mCRPC) remains an incurable malignancy.

In some embodiments, the presently disclosed subject matter can be used to target mCRPC by arming PBMC with anti-CD3 x anti-HER2 BiAb or other BiAb that targets prostate cancer antigens.⁵⁶ In some embodiments, the presently discloses subject matter enhances survival for hormone refractory prostate cancer patients who failed hormonal therapy.

Breast Cancer. An estimated 18,600 patients (pts) will die of breast cancer (BrCa) in 2021 and for those with metastatic BrCa (MBC), the median overall survival (OS) is between 18 and 24 months (mos).²⁵ Conventional chemotherapy (chemoT) for hormone receptor positive (HR+) human epidermal growth factor receptor 2 negative (HER2−) MBC after failure of endocrine therapy and >2 lines of chemoT is dismal. In phase III trials of ≥2 lines of chemoT for HER2-metastatic disease, the median time to progression (TTP) ranged from 3 to 6.3 mos.^{25,26,27,28,29,30} The median OS for HER2-MBC pts ranges from 8.1 to 12.9 mos which is usually reported with triple-negative breast cancer (TNBC) after 2-3 lines (L) of chemoT.³¹⁻³⁴

In some embodiments, the presently disclosed subject matter can treat HER2 0-3+ tumors in patients with fresh PBMC armed with anti-CD3 x anti-HER2 or anti-CD3 x anti-EGFR BiAbs.

Pancreatic Cancer. Conventional chemotherapy (chemo) for locally advanced pancreatic cancer (LAPC) or metastatic pancreatic cancer (MPC) is associated with dismal response rates and poor survival. An estimated 45,750 people will die of the disease in 2019 with a 5 year relative survival rate of only 9%.³⁵ In 1997, gemcitabine (gem) became first-line chemo for advanced pancreatic cancer (PC).³⁶ In 2013, the combination of nab-paclitaxel (nab) and gem improved overall survival (OS) from 6.7 months (mos) for gem alone to 8.5 mos for the combination.³⁷ FOLFIRINOX, a treatment regiment of advanced pancreatic cancer, improved the median OS to 11.1 mos.³⁸ OS after 2^{nd} line chemo is 6 mos compared to patients (pts) who receive supportive care.³⁹ Gem and platinum-based combinations provided a progression free survival (PFS) and OS of 4 and 6 mos, respectively.

In some embodiments, the presently disclosed subject matter can be used to treat metastatic or unresectable pancreatic cancer by arming fresh PBMC with anti-CD3 x anti-HER2 or anti-CD3 x anti-EGFR BiAbs.

Glioblastoma. Glioblastoma (GBM) is the most frequent type of primary malignant brain tumor, with an estimated 12,970 new cases for 2021 in the US.⁴⁰ The median overall survival (OS) for the general population is 8 months: 40 although for patients included in clinical trials is about 14-16 months.⁵⁵ Despite advances in treatment with maximum feasible surgical resection followed by radiation therapy (RT) and concurrent daily temozolomide (TMZ) followed by 6 cycles of adjuvant TMZ, the 5-year survival is less than 5%. 41 Patients with methylated 06-methylguanine-DNA methyl transferase (MGMT), an enzyme responsible for DNA repair have a median OS of 12.6 months: in contrast, patients with methylated MGMT have a median OS of 23.4 months.⁴²

In some embodiments, the presently disclosed subject matter can be used to treat patients with primary or recurrent glioblastomas by arming fresh PBMC with anti-CD3 x anti-HER2 or anti-CD3 x anti-EGFR BiAb.

Neuroblastoma/Osteosarcoma. High risk neuroblastoma (NB) is an aggressive and therapy resistant pediatric cancer where survival has remained unchanged for decades due to inability to eradicate residual disease even with most intensive chemotherapy regimens. Ganglioside GD2 is an established target for antibody dependent cellular cytotoxicity (ADCC) of NB.^{43,44} NK cells as well as granulocytes were found to be the main effectors of ADCC of NB cells.^{45,46} MAbs including murine 14G2a, 3F8, and chimeric ch14.18 were used in phase I/II studies and induced clinical responses in significant proportions of refractory and recurrent disease patients. ⁴⁷ Subsequently, high risk NB therapies have evolved to include GD2-directed mAb immunotherapy of minimal residual disease post autologous stem cell transplant. The role of immunotherapy in NB was established based on the outcomes of the randomized clinical study of ch14.18 mAb showing that the anti-GD2 mAb approach combined with ADCC-enhancing cytokines (IL-2 and GM-CSF) significantly improved progression free survival.⁴⁸

In some embodiments, the presently disclosed subject matter can be used to treat patients with refractory neuroblastoma or osteosarcoma by arming fresh PBMC with anti-CD3 x anti-HER2 or anti-CD3 x anti-GD2 BiAb. Indeed, any active agent for any disease, disorder, or other condition as disclosed herein can be employed on the other side of the anti-CD3.

Arming of fresh IECs (e.g., T cells such as (FBATs or other immune cells in PBMC with multispecific antibodies, such as bispecific antibodies (BiAbs), can overcome major barriers for successful adoptive immunotherapy. This approach takes the advantage of the targeting specificity of monoclonal antibodies and the cytotoxic capacity of T cells to lyse tumors. Arming with a multispecific antibody such as a BiAb makes IECs (e.g., T cell or other immune effector cells in PBMC) into an antigen-specific cytotoxic T lymphocyte (CTL) and infusions of such cells markedly increase the effective precursor frequency of CTL in a cancer patient. Furthermore, the armed fresh IECs (e.g., T cells or other immune effector cells) act as serial killers that proliferate without rearming with a multispecific antibody such as a BiAb, secrete tumoricidal cytokines, secrete chemokines, and survive in patients. Also, the infused cells immunized to the target cancer antigens as well as induce endogenous immune cells to become sensitized and immunized to the tumor. Fresh ICEs armed with a multispecific antibody (e.g., Fresh BiAb armed T cells (FBATs)) can act as a cytotoxic “drug”, kill multiple times (direct killing), divide after killing (increasing the effector: target (E: T) ratio in vivo), secrete tumoricidal cytokines to enhance endogenous immune responses, secrete chemokines at the tumor site to recruit naïve cells and antigen-presenting cells to immunize the patient to tumor lysate, and persist in patients to provide long-term anti-tumor immunity.

In one aspect of the presently disclosed subject matter, there is provided a method for treatment of a patient suffering from a cancer, an autoimmune disease, an infection, and/or a neurodegenerative disorder. In some embodiments, the disease or disorder is characterized by inflammation, which can be treated by a method, composition, or use as disclosed herein. In some embodiments, the method comprises (a) providing fresh IECs armed with one or more multispecific antibodies, wherein the multispecific antibodies are bound to the IECs without culture incubation; and (b) administering a composition of cells into the patient, wherein the composition of cells comprises a multispecific antibody bound IEC. In some embodiments, providing fresh IECs comprises isolating fresh IECs from the patient. Thus, in some embodiments, the method further comprises isolating fresh immune effector cells (IECs) from the patient. In some embodiments, the cells are administered back into the patient after arming, without culture incubation. Thus, the cells are autologous to the patient. In some embodiments, the armed IECs are cryopreserved. In some embodiments, providing the armed IECs thus comprises thawing and administering the thawed armed IECs to the subject. In some embodiments, the armed IECs can be allogenic to a subject. This can particularly be the case where the fresh IECs comprise polymorphonuclear neutrophils (PMNs). Since PMNs do not bear HLA antigens, they can be obtained from an unrelated blood bank donor. Further, any disease or disorder that would be benefited through treating a patient with a composition, use, or method in accordance with the presently disclosed subject matter as would be apparent to one of ordinary skill in the art upon a review of the instant disclosure falls within the scope of the presently disclosed subject matter. In some embodiments, the disease or disorder is characterized by inflammation, which can be treated by a method, composition, or use as disclosed herein.

In some embodiments, the presently disclosed subject matter provides a method of preparing a composition of cells comprising (a) isolating fresh immune effector cells (IECs) from a patient; and (b) arming the fresh IECs with one or more multispecific antibodies, wherein the multispecific antibodies are bound to the fresh IECs without culture. In some embodiments, the method further comprises freezing the composition of cells. In some embodiments, the method further comprises (c) infusing into a subject armed IECs without culture or cryopreservation.

In some embodiments, the presently disclosed subject matter provides a composition produced by the foregoing methods.

In some embodiments, the presently disclosed subject matter provides a composition comprising fresh immune effector cells (IECs) armed with one or more multispecific antibodies, wherein the one or more multispecific antibodies are bound to the fresh IECs.

In some embodiments, the presently disclosed subject matter provides a composition for use in treating a patient suffering from a cancer, an autoimmune disease, a neurodegenerative disorder, an infection and/or any combination thereof, wherein the composition comprises fresh immune effector cells (IECs) armed with one or more multispecific antibodies, wherein the multispecific antibodies are bound to the fresh IECs. In some embodiments, the disease or disorder is characterized by inflammation, which can be treated by a method, composition, or use as disclosed herein.

In some embodiments, the presently disclosed subject matter provides use of a composition comprising fresh immune effector cells (IECs) armed with one or more multispecific antibodies, wherein the one or more multispecific antibodies are bound to the fresh IECs for the manufacture of a medicament for treating a cancer, an autoimmune disease, a neurodegenerative disorder, an infection and/or combination thereof. In some embodiments, the disease or disorder is characterized by inflammation, which can be treated by a method, composition, or use as disclosed herein. In some embodiments, the fresh IECs comprises a cell selected from the group consisting of peripheral blood mononuclear cells (PBMC), an antigen-presenting cells. T cell (optionally fresh BiAb armed T cells (FBATs)), NK cells, monocytes, polymorphonuclear neutrophils (PMNs), and any combination thereof.

In some embodiments, the composition further comprises a pharmaceutically acceptable carrier, excipient, and/or diluent, optionally wherein the pharmaceutically acceptable carrier, excipient, and/or diluent is pharmaceutically acceptable for use in a human.

In some embodiments, the fresh IECs are isolated from a patient, optionally a patient to be treated. In some embodiments, the IECs are autologous or allogenic to the patient. In some embodiments, the composition comprises augmenting cytokines IL-2, IL-7, IL-15, IL-12, and/or GM-CSF and/or a checkpoint inhibitor.

In some embodiments, the fresh IECs comprise a cell selected from the group consisting of a peripheral blood mononuclear cell (PBMC), antigen-presenting cells, T cells (fresh BiAb armed T cells (FBATs)), NK cells, monocytes, polymorphonuclear neutrophils (PMNs) and any combination thereof. In some embodiments, the fresh IECs are T cells. In some embodiments, the fresh IECs are neutrophils obtained by buffy coats preparations from unrelated blood bank donors. In some embodiments, the fresh IECs are T cells, monocytes, or NK cells isolated from PBMCs.

In some embodiments, the method further comprises co-administering, e.g. co-infusing, a composition of antigen-presenting cells (APC), and said FBATs armed with multispecific antibody: wherein the APC and the FBATs are autologous to the patient, and the patients are given Interleukin (IL)-2, IL-7, IL-15, granulocyte-macrophage colony stimulating factor (GM-CSF) and/or check point inhibitors.

In some embodiments, the method further comprises administering, including co-administering, e.g., co-infusing a FBAT armed with the multispecific antibody: wherein the FBAT armed with the multispecific antibody are derived from an allogeneic donor. Since PMNs do not bear HLA antigens, they can be obtained from an unrelated blood bank donor.

In some embodiments, the IECs (e.g., FBATs) armed with the multispecific antibody are administered with a cytokine, chemokine, and/or check point inhibitor. In some embodiments, the cytokine or chemokine comprises IL-2, IL-7, IL-12, IL-15, GM-CSF and/or and combinations thereof. In some embodiments, the method comprises augmenting administration of the composition of cells by administering augmenting cytokines IL-2, IL-7, IL-15, IL-12, GM-CSF and/or check point inhibitors. In some embodiments, augmenting cytokines comprise IFN-gamma, TNFα, IP-10 (CXCL10), RANTES, CD40L (CD40 ligand) and/or MIP-α, which are co-administered, e.g., co-infused with IECs (e.g., FBATs). However, in some embodiments, if desired, the IEC is transduced with one or more vectors encoding a chemokine or cytokine, in accordance with art-recognized techniques.

In some embodiments, the check point inhibitor comprises an anti-PD1 monoclonal antibody. In some embodiments, the check point inhibitor comprises an anti-PD-L1 monoclonal antibody. In some embodiments, the check point inhibitor comprises an anti-CTLA-4 monoclonal antibody. In some embodiments, the check point inhibitors are selected from the group consisting of Nivolumab, Pembrolizumab, Cemiplimab, Dostarlimab, Durvalumab, Avelumab, Durvalumab, Ipilimumab and any combination thereof. In some embodiments, the checkpoint inhibitor is co-administered, e.g., co-infused with an armed ICE (e.g. FBATs).

In some embodiments, the multispecific antibodies comprise bispecific antibodies. In some embodiments, the bispecific antibody is selected from the group consisting of anti-CD3 x anti-HER2 (trastuzumab), anti-CD3x EGFR (cetuximab), anti-CD3 x anti-CD20 (ritixumab), anti-CD3 x anti-CS-1 (elotuzumab), Anti-CD3 x anti-GD2 (hu3F8) and any combination thereof. In some embodiments, the multispecific antibodies comprise trispecific antibodies. In some embodiments, the trispecific antibodies bind CD38, CD3 and CD28.⁵⁷ In some embodiments, the trispecific antibodies engage NK cells.⁵⁸ In some embodiments, the trispecific antibodies engage CD16, IL15 and CD33.⁵⁹ In some embodiments, the trispecific antibody engages CD19, CD20 and CD22.⁶⁰

Catumaxomab®(Removab) is an anti-CD3 x anti-Epcam with a functional FC portion that binds (hence trifunctional). In some embodiments, catumaxomab is used to arm ICEs (e.g., T cells) and augment ADCC via the FC binding to ADCC mediated NK/monocytes.

In some embodiments, the multispecific antibody comprises two monoclonal antibodies. In some embodiments, the multispecific antibody comprises a specificity for a tumor antigen. In some embodiments, the multispecific antibody comprises a specificity for a T cell receptor complex. In some embodiments, the multispecific antibody comprises specificities for a tumor antigen and a T cell receptor complex. In some embodiments, the multispecific antibody comprises specificities for an antigen selected from the group consisting of CD28, CD3, CD33, CD123, CD20, SLAMF7, BCMA, other liquid tumor antigens, HER2, EGFR, GD2, CEA, PSMA, PSA, VEGF, other solid tumor antigen present on a surface of a cancer cell and any combination thereof. In some embodiments, the multispecific antibody comprises monoclonal antibodies that are chemically heteroconjugated to form a bispecific antibody. In some embodiments, the multispecific antibody comprises a monoclonal antibody, a recombinant bispecific antibody and/or a bispecific antibody drug conjugate directed to any tumor associated antigen (TAA).

In some embodiments, the multispecific antibody comprises an antibody construct of a plurality of specificities, cytokine, or cytokine/chemokine receptors. In some embodiments, the multispecific antibody comprises an antibody construct having at least three functional components, wherein one is directed to the T cell receptor complex, one is directed to a TAA, and one comprises a cytokine or cytokine/chemokine receptor. In some embodiments, the multispecific antibody comprises an antibody construct having at least three functional components, wherein one is directed at the T cell receptor complex, one is directed at a TAA, and one comprises an antibody drug conjugate.

In some embodiments, the multispecific antibody comprises an antibody construct comprising at least 4 specificities or functional components, such as but not limited to one for the T cell receptor complex, one to one or more tumor antigen(s), an antibody drug conjugate, a cytokine, or a cytokine/chemokine receptor.

In some embodiments, the multispecific antibody comprises an anti T cell receptor monoclonal antibody component of the multispecific antibody directed against a CD3 of the T cell receptor complex.

In some embodiments, the patient that is immunosuppressed. In some embodiments, the patient is susceptible to, or suffering from a disease associated with autoimmune reactions.

In some embodiments, the method further comprises freezing the composition of cells. In some embodiments, the method further comprises thawing the composition of cells prior to administering, e.g., reinfusing the composition of cells into a patient in need of therapy. Whilst not wishing to be bound by theory, fresh IECs armed with multispecific antibodies, or the composition of cells may be frozen after the arming process for use at a later time where the fresh IECs armed with multispecific antibodies or the composition of cells will be thawed and reinfused into the patient. In some embodiments, the fresh IECs armed with multispecific antibodies, or the composition of cells can be autologous or allogenic to the patient.

In some embodiments, the composition of cells is configured as a dose administered to the patient. In some embodiments, the dose comprises an effective amount, e.g. a therapeutically effective amount, of armed IECs. In some embodiments, the IECs are armed with a dose of one or more multispecific antibodies of about 0.1 ng per million fresh IECs to about 1000 ng per million fresh IECs. In some embodiments the dose is optimized for each individual patient by titrating fresh and thawed aliquot of a frozen composition of cells to achieve a percent specific cytotoxicity level at an effector to target (E: T) ratio from about 10:1 to at least about 10% against a tumor target.

In some embodiments, the dose is at least about 5×10⁵ armed IECs per kilogram body weight of the patient with a dose schedule that can range from at least 4 infusions to 24 infusions. In some embodiments, the dose is at least about 0.5×10⁶ armed IECs per kilogram body weight of the patient with a dose schedule that can range from at least 4 infusions to 24 infusions. In some embodiments, the dose is at 40×10⁹ armed IECs per kilogram body weight of the patient with a dose schedule that can range from at least 4 infusions to 24 infusions. In some embodiments, the total infusion dose is at 320×10⁹ armed IECs per kilogram body weight of the patient. In some embodiments, the infusing dose is administered ranging from once a week to 1 once per month up to a year.

In some embodiments, the fresh IECs comprise CD4 positive cells. In some embodiments, the CD4 positive cells are selected form the group consisting of PBMCs, antigen-presenting cells, T cells (e.g., fresh BiAb armed T cells (FBATs)), NK cells, monocytes or polymorphonuclear neutrophils (PMNs). In some embodiments, the T cell comprises a CD3/CD4 positive cell. In some embodiments, the T cell comprises a CD3/CD8 positive cell.

In some embodiments, the fresh IECs comprise NK cells. In some embodiments, NK cells are selected from PBMCs and armed with multispecific antibody with one specificity directed at NK2D or FCγRIIIA (CD16) and the other directed at TAA.

In some embodiments, the fresh IECs comprise monocytes. In some embodiments, monocytes are selected from PBMCs and armed with multi-specific antibody with one specificity directed at FCγRIIIA CD14, of Fcγ RH (CD (4) and other directed at TAA

In some embodiments, the composition of cells is co-administered with other forms of therapy and/or antigen-presenting cells, immunocompetent T cells and immunocompetent naïve B cells.

In some embodiments, the method further comprising infusing intravenously or injecting into a tumor arterial supply or tumor site FBATs armed with bispecific antibody: wherein FBATs armed with bispecific antibody are derived from an autologous donor.

In some embodiments, the cellular composition reinfused into patients is free of soluble multispecific antibody.

In some embodiments, the cancer is selected from the group consisting of prostate cancer, breast cancer, leukemia, colon cancer, brain cancer, lung cancer, ovarian cancer, osteosarcoma, and neck cancer. In some embodiments, the composition of cells produce cytokines that promote an immune response in the patient. In some embodiments, the cytokines produced by the composition of cells recruit naïve T and B cells, NK cells, monocytes, or other immune response cells, to the site whereby said PBMC, FBATs, or other immune effectors are targeted.

In some embodiments, the PBMC, FBATS, or other IECs by-pass major histocompatibility restriction via the retargeting of the tumor specific antigen portion of the BiAb to the tumor.

In some embodiments, the method further comprises the composition of cells stimulating or inducing the development of antigen-specific memory T helper cells from naïve immune cells in said patient after reinfusing said composition of cells into said patient. In some embodiments, the antigen-specific memory T helper cells are Th1. In some embodiments, the antigen-specific memory T cells are directed to abnormal or tumor antigens. In some embodiments, the abnormal or tumor antigens are CD33, CD123, CD20, SLAMF7, BCMA, other liquid tumor antigens, HER2, EGFR, GD2, CEA, PSMA, PSA, VEGF), and/or other solid tumor antigens.

In some embodiments, the method further comprises said composition of cells stimulating or inducing the development of antigen-specific cytotoxic T cells from naïve immune cells in said patient after reinfusing said composition of cells into said patient. In some embodiments, the antigen-specific cytotoxic T cells are specific for abnormal or tumor antigens and where the antigen-specific cytotoxic T cells comprise Tc1 or Tc2. In some embodiments, the abnormal or tumor antigens are CD33, CD123, CD20, SLAMF7, BCMA, other liquid tumor antigens, HER2, EGFR, GD2, CEA, PSMA, PSA, VEGF, and/or other solid tumor antigens.

In some embodiments, the method further comprises said composition of cells inducing or stimulating the development of antigen-specific T cells directed to antigens other than the antigen targeted by the BiAb in said patient after reinfusing said composition of cells into said patient. In some embodiments, the method further comprises said composition of cells stimulating or inducing the development of antigen-specific memory B cells from naïve immune cells in said patient after reinfusing said composition of cells into said patient. In some embodiments, the method further comprises producing antibody specific for abnormal or tumor antigens in said B cells. In some embodiments, antigens are CD33, CD123, CD20, SLAMF7, BCMA, other liquid tumor antigens, HER2, EGFR, GD2, CEA, PSMA, PSA, VEGF, and/or other solid tumor antigens.

In some embodiments, the method further comprises said composition of cells inducing or stimulating the development of antigen-specific B cells directed to antigens expressed on the patient's tumor or abnormal cells after reinfusing said composition of cells into said patient.

In some embodiments, the method further comprises said composition of cells increasing the precursor frequency of antigen specific T cells after reinfusing said composition of cells into said patient. In some embodiments, the T cells are T helper cells. In some embodiments, the T cells are cytotoxic T cells. In some embodiments, the T cells are specific for CD33, CD123, CD20, SLAMF7, BCMA, other liquid tumor antigens, HER2, EGFR, GD2, CEA, PSMA, PSA, VEGF, and/or other solid tumor antigens. In some embodiments, the T cells are specific for abnormal or tumor antigens other than the targeted tumor antigen.

In some embodiments, the bispecific antibody, trispecific antibody, or other construct comprises a humanized monoclonal antibody, a human phage display library derived human antibody or a genetically engineered antibody. In some embodiments, the patient is immunosuppressed. In some embodiments, the autologous or allogeneic IECs (e.g., autologous or allogeneic T cells), are transduced with vectors coding for bispecific antibody, trispecific antibody, other genetically engineered antibodies, chemokines or cytokines in accordance with technique recognized in the art

In some embodiments, the multispecific antibodies are capable of inducing an immune response. In some embodiments, the method further comprises at least one of the following: (a) the composition of cells stimulating or inducing the development of antigen-specific memory T helper cells from naïve immune cells in the patient after reinfusing the composition of cells into the patient: (b) the antigen-specific memory T helper cells are Th 1 or Th2: (c) the antigen-specific memory T cells are directed to abnormal or tumor antigens: (d) the abnormal tumor antigens are HER2/neu: (e) the composition of cells stimulating or inducing the development of antigen-specific cytotoxic T cells from naïve immune cells in the patient after reinfusing said composition of cells into said patient: (f) the antigen specific cytotoxic T cells, Tc1 and Tc2, are specific for abnormal or tumor antigens: (g) said composition of cells inducing or stimulating the development of antigen-specific T cells directed to antigens other than the antigen targeted by the bispecific antibody after reinfusing said composition of cells into said patient: (i) said composition of cells stimulating or inducing the development of antigen-specific memory B cells from naïve immune cells after reinfusing said composition of cells into said patient: (j) said B cells produce antibody specific for abnormal or tumor antigens: (k) said composition of cells inducing or stimulating the development of antigen-specific B cells directed to antigens expressed on the patient's tumor or abnormal cells after reinfusing said composition of cells into said patient: (1) said composition of autologous cells increasing the precursor frequency of antigen specific T cells in said patient after reinfusing said composition of cells into said patient: (m) said T cells are T helper cells: (n) said T cells are cytotoxic T cells: (o) said T cells are specific for the targeted tumor antigen(s): or (p) said T cells are specific for abnormal or tumor antigens other than the targeted tumor antigen(s).

In some embodiments, the antigen-specific IECs (e.g., PBMC or FBATs) survive, proliferate and give rise to memory T cells after contact with abnormal or tumor antigens. In some embodiments, the memory T cells are T helper cells.

In some embodiments, the memory T cells are cytotoxic T cells. In some embodiments, the patient is a mammal. In some embodiments, the patient is suffering from, or susceptible to disease characterized by abnormal cell growth and proliferation. In some embodiments, the patient is suffering from, or susceptible to disease characterized by autoimmunity. In some embodiments, the patient is suffering from a neurodegenerative disorder. In some embodiments, the wherein the patient is suffering from a viral, bacterial, or fungal infection.

In some embodiments, the IECs undergoing cell division in vivo.

In some embodiments, freshly armed IECs (e.g. PBMC, T cells, NK cells, PMNs, or monocytes) undergoing multiple cycles of antigen specific tumor cell killing.

In one aspect, the presently disclosed subject matter provides a method for treatment of a patient suffering from cancer. In some embodiments, the method comprises the steps of: (a) isolating a sample of peripheral blood mononuclear cells, comprising T cells, from a patient suffering from cancer: (b) arming one or more of said T cells with BiAb: (c) arming of said T cells with BiAb capable of binding to the T cell receptor complex of a T cell, and to tumor-associated antigens on a tumor cell, under conditions wherein: (i) said bispecific antibody binds to said T cells, tumor cells, and Fc receptor positive cells, (ii) said antibody binds to the tumor target and said antibody binding to said tumor target activates said T cells, (iii) said antibody redirects said T cells and Fc-receptor positive cells to said tumor cells, (iv) said bispecific antibody activated T cells destroy said tumor cells; and, (d) reinfusing, into the patient, a composition of autologous cells comprising said bispecific antibody armed activated T cells (FBAT) as treatment of the patient wherein: (i) contacting T cells specific for different epitopes on the tumor cell with the autologous cells and including proliferation of the T cells specific for different epitopes on the tumor cells, or targeting and contacting multiple target cells with the armed T cell to which the BiAb remains bound, and killing said multiple target cells.

In one aspect, the presently disclosed subject matter provides a method of treatment. In some embodiments, the method comprises (a) providing a formulation comprising (i) a pharmaceutically acceptable excipient; and (ii) PBMC having bound thereto a bispecific antibody with a binding specificity for a T-cell antigen, selected from the group consisting of CD28 and CD3, and a binding specificity for a CD33, CD123, CD20, SLAMF7, BCMA, other liquid tumor antigens, HER2, EGFR, GD2, CEA, PSMA, PSA, VEGF, and/or other solid tumor antigen present on a surface of a cancer cell, wherein the bispecific antibody has a binding specificity of about 10-8 moles/liter or higher: (b) infusing said formulation into a human patient suffering from cancer: (c) binding the bispecific antibodies to cancer cells in the patient: (d) contacting the cancer cells in the patient with the PBMCs and lysing the cancer cells in the patient: (e) creating a T cell memory in the patient's endogenous T cells; and (f) contacting cancer cells in the patient with the patient's endogenous T cells and lysing said cancer cells.

In one aspect, the presently disclosed subject matter provides a method of preparing a composition of cells. In some embodiments, the method comprises (a) isolating fresh immune effector cells (IECs) from the patient and (b) arming the fresh IECs with one or more multispecific antibodies, wherein the multispecific antibodies are bound to the fresh IECs without culturing. In some embodiments, the method further comprises freezing the composition of cells.

In one representative embodiment, the presently disclosed subject matter combines the non-MHC restricted cytotoxicity mediated by fresh (uncultured and/or without ex vivo expansion) IECs, such as peripheral blood mononuclear cells that contain T cells, T cell subsets, natural killer (NK), polymorphonuclear neutrophils (PMNs) or other IECs and the specificity of bispecific antibodies (BiAb) to enhance the cytotoxicity of T cells to target and lyse tumor cells.

In some embodiments, the presently disclosed subject matter provides a method for treating a cancer in a subject in need thereof. In some embodiments, the method comprises administering to the subject an effective amount of a composition comprising a fresh (uncultured)bispecific antibody armed T Cells (FBAT) in the whole population of fresh PBMC, which selectively binds a cell of the cancer in the subject, to thereby treat the cancer in the subject. In some embodiments, the FBAT is selected from the group comprising a bispecific antibody (BiAb) armed fresh T cell (FBAT) and fresh tumor infiltrating lymphocyte. The method further arms fresh NK cells, fresh monocyte, fresh PMNs, and B cells with BiAb or antibodies to engage cancers of hematologic origin and solid tumors.

In some embodiments, the presently disclosed subject matter further provides compositions and methods for treatment of tumors on an individual patient basis by arming fresh autologous or allogeneic T cells with BiAb, multiple BiAbs, BiAb drug conjugate or other BiAb or Trispecific constructs specific for tumor antigen(s). The presently disclosed subject matter is unexpected from the prior art. Prior art describes arming of ex vivo expanded T cells, gene transduction (lente or retroviral vectors) of activated T cells with single chain fragment variable region contracts scFV CAR-T cells, CAR-T armed with BiAbs (Published U.S. Patent Application No. US2018/0243341A1 to June et al., published Aug. 30, 2018: Published U.S. Patent Application No. US20180282693A1 to June et al., published Oct. 4, 2018: Archana Thakur, John Scholler, Ewa Kubicka, Edwin T. Bliemeister, Dana L. Schalk, Carl H. June, Lawrence G. Lum Front Immunol. 2021:12: 690437. Published online 2021 Jul. 5. doi: 10.3389/fimmu.2021.690437 PMCID: PMC8288104 and Archana Thakur, John Scholler, Dana L. Schalk, Carl H. June, Lawrence G. Lum J Cancer Res Clin Oncol. Author manuscript: available in PMC 2021 August 1. Published in final edited form as: J Cancer Res Clin Oncol. 2020 August: 146 (8): 2007-2016. Published online 2020 May 24. doi: 10.1007/s00432-020-03260-4: PMCID: PMC7375514), naked CAR-T (with scFV) or headless CARs, (Published U.S. Patent Application No. US2018/0243341A1 to June et al., published Aug. 30, 2018: Published U.S. Patent Application No. US20180282693A1 to June et al., published October 4. 2018: Archana Thakur, John Scholler, Ewa Kubicka, Edwin T. Bliemeister, Dana L. Schalk, Carl H. June, Lawrence G. Lum Front Immunol. 2021:12: 690437. Published online 2021 Jul. 5. doi: 10.3389/fimmu.2021.690437 PMCID: PMC8288104 and Archana Thakur, John Scholler, Dana L. Schalk, Carl H. June, Lawrence G. Lum J Cancer Res Clin Oncol. Author manuscript: available in PMC 2021 Aug. 1. Published in final edited form as: J Cancer Res Clin Oncol. 2020 August: 146 (8): 2007-2016. Published online 2020 May 24. doi: 10.1007/s00432-020-03260-4; PMCID: PMC7375514), TILs or NK cells after culture with BiAb or infusions of BiAb alone directly into the patient(s) without ex vivo arming of the activated, and/or cultured immune effectors cells. In some embodiments, the presently disclosed subject matter bypasses any need to ex vivo culture of the immune effector cells in the peripheral blood product prior to a therapeutic infusion into patients. In some embodiments, the BiAb construct replaces the use of OKT3 and IL-2 or anti-CD3/anti-CD28 beads and IL-2 used to trigger growth, proliferation, killing, and cytokine secretion by directly inducing growth, proliferation, killing, and cytokine secretion. In some embodiments, this approach bypasses anti-CD3+IL-2 or anti-CD3/anti-CD28+IL-2 but using the anti-CD3 portion of the BiAb to activated T cells for subsequent expansion and CTL activity in vivo.

In general, the presently disclosed subject matter provides for the arming IECs, such as PBMC that contain T cells, NK cells, monocytes, polymorphonuclear cells, and B cells, with a BiAb that binds to T cells or other immune effectors on one hand and to tumor associated antigens on the other hand. The present approach is advantageous in many aspects, including that it combines the specificity of the antibody directed at immune effector cells such CD3 on T cells, NKG2D on NK cells, Fc-gamma (CD64, CD16, CD32) on T cells, NK cells, monocytes, CD89 receptors on PMNs, on T cells, monocytes, or B cells and a tumor associated antigen(s) to augment the cytotoxic capacity of the immune cells to lyse TAA or hematologic malignancies. For example, the BiAb bridge between the fresh T cells and the Her2+ target redirects the cytotoxicity of T cells to the Her2+ targets, while bypassing major histocompatibility restrictions. The presently disclosed subject matter can bypass the toxic side effects of co-administered chemokines or checkpoint inhibitors. The freshly armed IECs (e.g., PBMC) kill AML blasts, breast and pancreatic carcinoma cell lines. The presently disclosed subject matter provides for immunotherapy comprising multiple infusions of bispecific antibody armed PBMC by itself or in combination with checkpoint inhibitors, irradiation, chemotherapies, or metabolic inhibitors or other anti-cancer agents including cancers that are drug resistant cancer or drug sensitive cancer. In some embodiments, the cancer is selected from the group comprising pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

In some embodiments, the IECs (e.g., FBAT) are prepared using and can comprise bispecific antibodies:
1) That target T cells: anti-CD3 x anti-EGFR BiAb, anti-CD3 x anti-HER2 BiAb, anti-CD3 x anti-GD2 BiAb, anti-CD3 x anti-CD20 BiAb, anti-CD3 x anti-CD30, anti-CD3 x anti-SLAMF7 BiAb, anti-CD3 x anti-BCMA, and anti-CD3 x anti-VEGF.
2) that target Fc-IgG bearing immune cells via CD64, CD32, and CD64 (expressing immune cells (NK cells, monocytes, PMNs): anti-Fc-IgG x anti-EGFR BiAb, anti-Fc-IgG x anti-HER2 BiAb, anti-Fc-IgG x anti-GD2 BiAb, anti-Fc-IgG x anti-CD20 BiAb, anti-Fc-IgG x anti-CD30, anti-FclgG x anti-SLAMF7 BiAb, anti-Fc-IgG x anti-BCMA, and anti-Fc-IgG x anti-VEGF.
3) that target Fc-IgA (CD89) on immune cells: anti-CD89 x anti-EGFR BiAb, anti-CD89 x anti-HER2 BiAb, anti-CD89 x anti-GD2 BiAb, anti-CD89 x anti-CD20 BiAb, anti-CD89 x CD30 BiAb, anti-CD89 x anti-SLAMF7, anti-CD89 x anti-BCMA, and anti-CD89 x anti-VEGF.

In some embodiments, the arming agent, also referred to in some embodiments, as a multispecific antibody, in some embodiments as a bispecific antibody or BiAb, used to arm the targeted activated cell is a chemically heteroconjugated BiAb, a recombinant BiAb of any configuration, a BiAb engineered with a nanoparticle payload, BiAb engineered with a chemically heteroconjugated trispecific antibody, BiAb or trispecific antibody (TriAb) drug conjugates, BiAb or TriAb drug with nanoparticles containing drugs or cytokines/chemokines, bispecific antibody cytokine constructs, and BiAb cytokine receptor constructs.

In some embodiments, the fresh IECs (e.g., PBMC, T cells or T cells subsets) are produced from an apheresis product by arming with a multispecific antibody, in some embodiments a bispecific antibody or BiAb stimulation in the presence of or absence of IL-2 (range of about 20) to about 3000) IU/ml). In some embodiments, PBMC armed with BiAb are costimulated with anti-CD3/anti-CD28 beads. The beads are removed after no more than 48 hours after stimulation. In this embodiment, the ex vivo expanded cultures of IECs (e.g., PBMC, T cells, or T cell subsets, or other immune cells) are harvested for and co-administered with freshly armed IECs. The IECs (e.g., BiAb stimulated PBMC or BiAb armed co-stimulated PBMC or purified T cells or T cell subsets) are harvested and infused or cryopreserved in aliquots to be infused later.

In some embodiments, the presently disclosed subject matter provides a method for treating a cancer, autoimmune disease, graft-rejection, graft-vs-host disease, infections (viral, bacterial, and fungal), and parasitic infections in a subject in need thereof.

If the tumor antigen changes in a patient, the multispecific antibody (e.g., BiAb or BiAb construct) used to arm the IEC (e.g., PBMC or subpopulations) can be replaced with different multispecific antibody (e.g., BiAb or BiAb construct) that is specific for the new antigen(s). It is expected that long term tumor specific/antigen-specific T cells and tumor specific antibodies are generated by the methods and compositions.

By way of example and not limitation, the presently disclosed subject matter is advantageous in fresh IEC (e.g., PBMC, T cells in PBMC) armed with a multispecific antibody (e.g., BiAb or BiAb construct) significantly increase the chances of overcoming barriers for successful adoptive immunotherapy by: 1) removing the need for ex vivo expanded (in vitro cultures) T cells, tumor infiltrating lymphocytes (TILs), NK cells, or other immune populations, prior to arming prior to reinfusion into the patient: 2) direct induction or activation of IEC leading to proliferation of effector cells in the patient using the patient as their own bioreactor: 3) induction of immediate and ongoing cytotoxicity mediated by the proliferating effector cells (e.g., T cells) that bind, target, and lyse tumor cells by immediately increasing the potency of the FBATs or other immune effector cells: 4) taking advantage of the ability of fresh IEC (e.g., T cells) to kill and produce large quantities of Th1 cytokines and immune attractive chemokines leading to more potent vaccination of the patients against their tumor antigens: 5) cytokine release syndrome will be self-limiting since there are fixed amount of multispecific antibody (e.g., BiAb or BiAb construct) franked onto the (IEC, e.g., T cells) that dilute out as a function of T cell proliferation: 6) the arming dose of arming agent, e.g., multispecific antibody, e.g., BiAb or multiple BiAbs, can be adjusted to increase or decrease the potency or side effects: 7) enhanced trafficking of freshly armed IEC (e.g., BiAb armed T cells) trafficking into tumor; and 8) enhanced secretion of cytokines with release of tumor associated antigens in the tumor microenvironment that induce endogenous immune cells to become immunized to the tumor antigens release during lysis process.

Another advantage of the presently disclosed subject matter is that only nanogram amounts of arming agent, e.g., multispecific antibody (e.g., multiple BiAb or TriAb) are needed to induce proliferation and specific cytotoxicity directed at the freshly armed IEC. The nanogram amounts of arming agent markedly decreases the chance of developing antibodies to the multispecific antibody construct thereby improving the safety of infusions. The use of autologous patient T cells or other immune cells will avoid any graft-versus-host-reaction disease, graft rejections, other autoimmune reactions and complications. Allogeneic fresh T cells armed with BiAb used to treat patient may not cause GVHD. For example, activated donor T cells after polyclonal activation with anti-CD3 would potentially preclude expansion of T cells in response to alloantigens by triggering all existing clones to respond prior to exposure to alloantigen. Therefore, the alloresponsive clones in the naïve T cell population would not have a change to respond in a mixed lymphocyte culture (MLC) stimulated by HLA antigens or in patients after infusion.

In some embodiments, a patient suffering from cancer is treated according to a method of the presently disclosed subject matter which comprises the steps of isolating IEC from the patient, such as the patient's PBMC. The cytotoxic activity is tested in vitro against tumor cells and an appropriate arming dose of a multispecific antibody, such as a BiAb, is determined, based on the cytotoxic activity. In some embodiments, the patient is then administered IEC, e.g. PBMC or immune subsets thereof, armed with a BiAb or multispecific antibody construct.

In some embodiments, PBMC which include fresh T cells are armed with at least about 0.5 ng antibody per million PBMCs, more preferably at least about 10 ng antibody per million PBMC, most preferably to at least about 200 ng antibody per million T cells. The arming dose, however, is optimized for each individual multispecific antibody (e.g., BiAb) construct by titrating PBMC and activated T cells from the same donor to determine the optimal specific cytotoxicity level at low effector to target ratios ranging from 10:1 to 0.5:1 at least about 10% above background against a tumor target.

In some embodiments, the dose of armed IECs is preferably about 0.5-1.0×10⁶ per kilogram body weight to 50×10⁶ per kilogram body given once per week up to once every two weeks. In one aspect of the presently disclosed subject matter, the patient receives at least about one infusion, optionally about eight infusions, further optional example schedules of multiple infusions: (a) once a week for 16 infusions: (b) one every 2 weeks for 16 infusions: (c) one a week for 4 weeks, then once every 2 weeks, and then 1 per month: (d) once every two weeks for about 16 infusions; and/or (e) combination of frequencies derived from clinical responses for specific diseases for optimal dosing frequencies. In some embodiments, the dose is at least about 5×10⁵ armed IECs per kilogram body weight of the patient with a dose schedule that can range from at least 4 infusions to 24 infusions. In some embodiments, the dose is at least about 0.5×10⁶ armed IECs per kilogram body weight of the patient with a dose schedule that can range from at least 4 infusions to 24 infusions. In some embodiments, the dose is at 40×10⁹ armed IECs per kilogram body weight of the patient with a dose schedule that can range from at least 4 infusions to 24 infusions. In some embodiments, the total infusion dose is at 320×10⁹ armed IECs per kilogram body weight of the patient. In some embodiments, the infusing dose is administered ranging from once a week to 1 once per month up to a year.

In some embodiments, the armed IEC is either a CD3/CD8 positive and/or a CD3/CD4 positive cell. In one aspect of the presently disclosed subject matter the IEC from a patient can be co-administered with other forms of therapy such as IL-2, IL-7, IL-15, pembro or other checkpoint inhibitors. The autologous T cells can be transduced with vectors coding for cytokines and/or chemokines or monoclonal or multispecific antibody thereby producing a high concentration of localized of chemokines, monoclonal or multispecific antibody.

In some embodiments, patients which have been treated with armed IECs induces specific anti-tumor cellular and humoral immune memory to the specific antigen recognized by the armed IECs. Memory cells are easily identifiable, e.g. by flow cytometric analysis (TCR and Interferon gamma cytoplasmic staining) or Interferon γ EliSpots in response to tumor cell lines bearing the target antigen(s).

In some embodiments, fresh armed IECs (e.g., PBMC, T cells or other immune effectors) are capable of killing multiple targets. In other words, the same freshly armed IECs (e.g., PBMC, T cells or other immune effectors) can target and kill cells, such as tumor cells, while proliferating after infusion into the patient.

In some embodiments, fresh armed IECs (e.g., T cells or other immune effectors), as a result of targeting tumor, develop into antigen-specific T cell clones directed at antigens on the tumor. For example, targeting HER2/neu induces the development of HER2 specific clones (that the bispecific antibody was directed at) as well as other epitopes on the HER2 receptor and other antigens on the tumors such as EGFR and/or CEA. Optionally, the induction of antigen specific clones directed at a specific tumor antigen allows for the maturation of the cellular and humoral response to induce the production of T cells that recognize antigens on the tumor that are yet undefined and unknown and B cells that produce specific antibodies to other epitopes of HER2 as well as other tumor associated antigens.

In one aspect of the presently disclosed subject matter, there is provided a method for treatment of a patient suffering from cancer, autoimmune disease, or neurodegenerative disorder. In some embodiments, the method comprises the steps of: (a) isolating a sample of immune effectors, such as but not limited to peripheral blood mononuclear cells (PBMC), comprising T cells (fresh BiAb armed T cells-(FBATs) or NK cells, monocytes, or polymorphonuclear neutrophils (PMNs) (referred to collectively as “immune effectors”, immune effector cells” or “IECs” or “IEC”): (b) arming of said fresh IECs (e.g., T cells or other immune effectors) with bispecific antibodies, trispecific antibodies, or other constructs capable of binding to a receptor complex of an immune effector, such as a T cell receptor complex of a T cell, and to an antigen on a cell, such as a tumor-associated antigen on a tumor cell, under conditions wherein: (i) said bispecific antibody, trispecific antibody, or other construct binds to said immune effectors and cells, such as FBATs and tumor cells, and/or immune effector cells and tumor cells, (ii) said bispecific antibody, trispecific antibody, or other construct binds to the target, such as the tumor target and said antibody or other construct binding to the target, such as the tumor target activates said immune effectors such as T cells, (iii) said bispecific antibody, trispecific antibody, or other construct redirects said FBATSs and/or immune effector cells to said target, such as tumor cells, (iv) said FBATs and/or immune effector cells to destroy said tumor cells; and, (v) reinfusing a composition of cells comprising said FBATs and/or said immune effector cells armed with said bispecific antibody, trispecific antibody, or other construct into the patient as treatment for the patient, and optionally either: (i) contacting T cells specific for different epitopes on the target, such as the tumor cell, with the composition of cells and inducing proliferation of the T cells specific for different epitopes on the target, such as the tumor cell, or (ii) targeting and contacting multiple target cells with the armed T cell to which the bispecific antibody, trispecific antibody, or other construct remains bound and killing said multiple target cells.

In some embodiments, the method further comprises co-infusing intravenously or co-injecting into a tumor arterial supply or tumor site a composition of antigen-presenting cells (APC), and freshly armed IECs or composition of cells (e.g., said FBATs armed with bispecific antibody); wherein said APC and said armed IECs are autologous or allogeneic to the patient, and patients are given IL-2, IL-7, IL-15, and/or GM-CSF during the therapy.

In some embodiments, a composition of PBMC, T cell, or T cells subsets, or other immune effectors are armed with a bispecific antibody and augmenting cytokines IL-2, IL-7, IL-15, IL-12, or GM-CSF or other immune augmenting cytokines are reinfused into a patient in need of such therapy.

In some embodiments, the method further comprises co-infusing intravenously or co-injecting into a tumor arterial supply or tumor site said armed IECs (e.g., FBATs armed with bispecific antibody): wherein said armed IECs (e.g., FBATs armed with bispecific antibody) are derived from an allogeneic donor and wherein a composition of allogeneic cells comprising said armed IECs (e.g., FBATs armed with said bispecific antibody) with or without IL-2, IL-7, IL-12, IL-15, and/or GM-CSF or other immune augmenting cytokines are reinfused into the patient in need of such therapy.

In some embodiments, the bispecific antibody comprises two monoclonal antibodies. In some embodiments, each of the specificities of said bispecific antibody are directed to a tumor antigen and the T cell receptor complex. In some embodiments, the bispecific antibody, trispecific antibody, or other construct comprises monoclonal antibodies that are chemically heteroconjugated to form the bispecific antibody. In some embodiments, the bispecific antibody comprises monoclonal antibodies directed to any tumor associated antigen. In some embodiments, the bispecific antibody is a recombinant bispecific antibody directed to any tumor associated antigen. In some embodiments, the bispecific antibody is a bispecific antibody drug conjugate directed to any tumor associated antigen. In some embodiments, the bispecific antibody is an antibody construct that has 2 specificities and contains a cytokine/chemokine receptor.

In some embodiments, the trispecific antibody is an antibody construct that has 3 specificities, one of which is the T cell receptor complex. In some embodiments, the trispecific antibody is an antibody construct that has 3 specificities and contains an antibody drug conjugate. In some embodiments, the trispecific antibody is an antibody construct that has 3 specificities and contains a cytokine/chemokine receptor. In some embodiments, the anti T cell receptor monoclonal antibody component of an bispecific antibody is directed against CD3 of the T cell receptor complex.

In some embodiments, the patient is immunosuppressed. In some embodiments, the patient is susceptible to, or suffering from diseases associated with abnormal cellular proliferation or growth. In some embodiments, the patient is susceptible to, or suffering from diseases associated with autoimmune reactions.

In some embodiments, the method further comprises freezing and thawing the armed IECs prior to reinfusing the armed IECs into the patient in need of such therapy.

In some embodiments, the IECs are armed with a multispecific antibody dose of about 1.0 ng per million fresh IECs to about 1000 ng per million fresh IECs. In some embodiments, IECs are armed with a bispecific antibody dose of about 1.0 ng per million fresh IECs to about 500 ng per million fresh PBMC or T cells. In some embodiments, IECs are armed with a bispecific antibody dose of about 0.1 ng per million fresh IECs to about 500 ng per million fresh PBMC or T cells. In some embodiments, the dose is optimized for each individual patient by titrating fresh and thawed aliquot of frozen armed IECs to achieve a percent specific cytotoxicity level at an effector to target ratio from about 10:1 to at least about 10% against the tumor target. In some embodiments, the dose administered to the patient is at least about 0.5×10⁶ armed IECs per kilogram body weight of the patient with doses that can range from a minimum of 4 infusions to a total of 24 infusions ranging from once a week to 1 once per month up to a year. In some embodiments, the dose administered to the patient ranges up to 1×10⁹ armed IECs per kilogram body weight of the patient with doses that can range from a minimum of 4 infusions to a total of 24 infusions ranging from once a week to 1 once per month up to a year. In some embodiments, the dose is at least about 5×10⁵ armed IECs per kilogram body weight of the patient with a dose schedule that can range from at least 4 infusions to 24 infusions. In some embodiments, the dose is at least about 0.5×10⁶ armed IECs per kilogram body weight of the patient with a dose schedule that can range from at least 4 infusions to 24 infusions. In some embodiments, the dose is at 40×10⁹ armed IECs per kilogram body weight of the patient with a dose schedule that can range from at least 4 infusions to 24 infusions. In some embodiments, the total infusion dose is at 320×10⁹ armed IECs per kilogram body weight of the patient. In some embodiments, the infusing dose is administered ranging from once a week to 1 once per month up to a year.

In some embodiments, the IECs include PBMC which contain T cells and other immune effectors. In some embodiments, the T cell is a CD3/CD4 positive cell. In some embodiments, the T cell is a CD3/CD8 positive cell.

In some embodiments, the armed IECs (e.g., PBMC, FBATs, or other immune effector cells) are co-administered with other forms of therapy and/or antigen-presenting cells, immunocompetent T cells and immunocompetent naïve B cells.

In some embodiments, the method further comprises infusing intravenously or injecting into a tumor arterial supply or tumor site armed IECs (e.g., FBATS armed with bispecific antibody): wherein armed IECs are derived from an autologous donor. In some embodiments, the method further comprises infusing intravenously or injecting into a tumor arterial supply or tumor site said armed IECs: wherein armed IECs are derived from an allogeneic donor.

In some embodiments, a composition of allogeneic cells comprising armed IECs (e.g., FBATS armed with bispecific antibody) are reinfused into a patient in need of such therapy in the presence or absence of IL-2, IL-7, IL-15, IL-17 or other immune augmenting cytokines.

In representative embodiments, the cellular composition reinfused into patients is free of soluble bispecific antibody. In some embodiments, the co-activated IEC (e.g., T cell) is armed with a bispecific antibody dose of about 0.1 ng per million IECs to about 500 ng per million IECs. In some embodiments, a dose to be administered to the patient is at least about 0.5×10⁶ armed IECs/kg body weight. In some embodiments, the patient receives at least about four infusions.

In some embodiments, the tumor is selected from the group consisting of prostate cancer, breast cancer, leukemia, colon cancer, brain cancer, lung cancer, ovarian cancer, osteosarcoma, and neck cancer.

In some embodiments, the armed IECs produce cytokines that promote an immune response in the patient. In some embodiments, the cytokines produced by the armed IECs comprise an interferon, granulocyte-macrophage colony stimulating factor, interleukin 2, or another interleukin, TNFα, RANTES, or MIP-α. In some embodiments, the cytokines produced by the armed IECs recruit naïve T and B cells, NK cells, monocytes, or other immune response cells, to the site whereby the armed IECs are targeted.

In some embodiments, a composition of autologous cells comprising the armed IECs with or without IL-2, IL-7, IL-15, IL-17, IL-12, GM-CSF or other immune augmenting cytokines or checkpoint inhibitor (anti-PD1 or anti-PDL1 monoclonal antibody) are reinfused into a patient in need of such therapy. In some embodiments, the bispecific antibody comprises two monoclonal antibodies. In some embodiments, the bispecific antibody comprises specificity directed to an antigen. In some embodiments, the bispecific antibody comprises specificities directed to antigens. In some embodiments, the specificities of said bispecific antibody are directed to a tumor antigen and the T cell receptor complex.

In some embodiments, the method further comprises targeting and contacting multiple target cells, which the armed T cell, to which the bispecific antibody remains bound, recognizes by recognizing the desired antigens on the target cells, and killing said multiple target cells.

In some embodiments, the method of treatment further comprises infusing a cytokine into the patient wherein the cytokine is chosen from IL-2, IL-7, IL-12, IL-15, IL-17 and GM-CSF. In some embodiments, the method of treatment further comprises inducing or stimulating the development of antigen-specific T cells directed to antigens other than the antigen targeted by the bispecific antibody, after reinfusing said composition of cells or armed IECs (e.g., autologous or allogenic cells) into said patient.

In some embodiments, the method of treatment further comprises (i) contacting T cells specific for different epitopes on the cancer cells, with the said composition of cells or armed IECs (e.g., autologous or allogenic cells) and inducing proliferation of the T cells specific for different epitopes on the tumor cell, or (ii) targeting and contacting multiple cancer cells with the T cell to which the bispecific antibody remains bound, and killing said multiple cancer cells.

In some embodiments, the method of treatment further comprises contacting natural killer cells with the said composition of cells or armed IECs (e.g., autologous or allogenic cells) and inducing proliferation of the natural killer cells.

In some embodiments, the T cells secrete immune activating cytokines and chemokines. In some embodiments, the T cells secrete MIP-1a or RANTES.

In some embodiments, the multispecific antibody, e.g., bispecific antibody, is directed against CD3 or specific tumor antigen. In some embodiments, the anti-CD3 antibody comprises OKT3 or equivalent recombinant construct in a multispecific, e.g., bispecific antibody. In some embodiments, an immune cells receptor is Fc-IgA receptor (CD89), CD45, FclgGRIIIb (CD16), FclgGRIIB (CD32), FclgGRI (CD64) or other receptors of immune effector cells.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, New York, United States of America: Glover (1985) DNA Cloning: A Practical Approach. Oxford Press, Oxford: Gait (1984) Oligonucleotide Synthesis: A Practical Approach, IRL Press, Oxford, England: Harlow & Lane, 1988, Antibodies, A Laboratory Manual, Cold Spring Harbor Publications, New York: Roe et al. (1996) DNA Isolation and Sequencing: Essential Techniques, John Wiley, New York, New York, United States of America; and Ausubel et al. (1995) Current Protocols in Molecular Biology, Greene Publishing.

### III. Examples

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Anti-CD3 x anti-CD123 bispecific antibody (CD123Bi) and anti-CD3 x anti-CD33GO (gemtuzumab ozogamicin [GO])bispecific antibody (CD33GOBi) were used to arm ATC to produce Bispecific antibody Armed activated T cells (designated CD123 BATs or CD33GO BATs) to target AML cell lines and peripheral blood mononuclear cells (PBMC) from AML patients and in vivo in xenogeneic NSG mice engrafted with leukemic cells.

Production of Anti-CD3 x Anti-CD123 and Anti-CD3 x anti-CD33GO Bispecific Antibodies. OKT3, a murine anti-CD3 epsilon IgG2a monoclonal antibody (mAb), was purchased from Miltenyi Biotech (Auburn, CA) and BioXCell (Lebanon, NH). Anti-CD123 (Clone 7G3) was purchased from BD Biosciences (NJ). Gemtuzumab ozogamicin (GO, Mylotarg™) a recombinant humanized anti-CD33 monoclonal antibody (IgG4 K antibody hP67.6) covalently linked to the calicheamicin (anti-CD33GO) was purchased from the UVA pharmacy (Charlottesville, Virgina). OKT3 was chemically heteroconjugated with anti-CD123 mAb and anti-CD33GO as described by Thakur A. Kondadasula S V, Ji K, Schalk D L, Bliemeister E, Ung J et al. Anti-tumor and immune modulating activity of T cell induced tumor-targeting effectors (TITE). Cancer Immunol Immunother 2021: 70 (3): 633-656. In brief, OKT3 was crosslinked using a 10-fold molar excess of Traut's reagent and anti-CD123 mAb or Gemtuzumab were crosslinked using a four-fold molar excess of Sulpho-SMCC. The BiAb was produced by combining crosslinked mAbs at a 1:1 ratio by overnight heteroconjugation at 4° C. to produce anti-CD3 x anti-CD123 BiAb (CD123Bi) and anti-CD3 x anti-CD33GO BiAb (CD33GOBi).

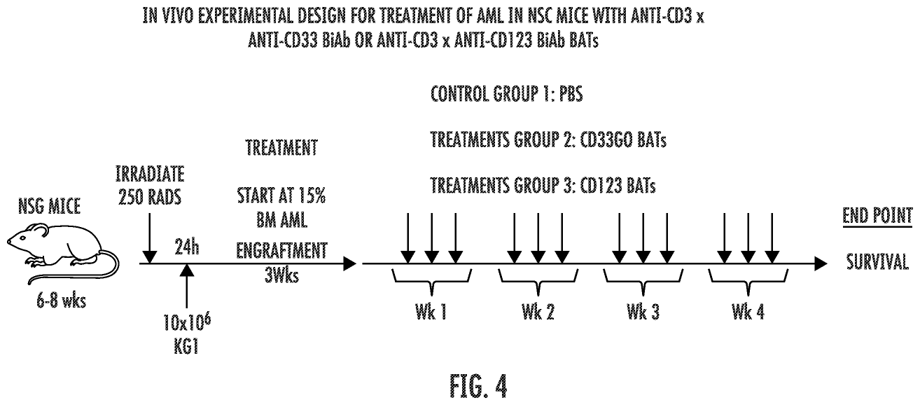

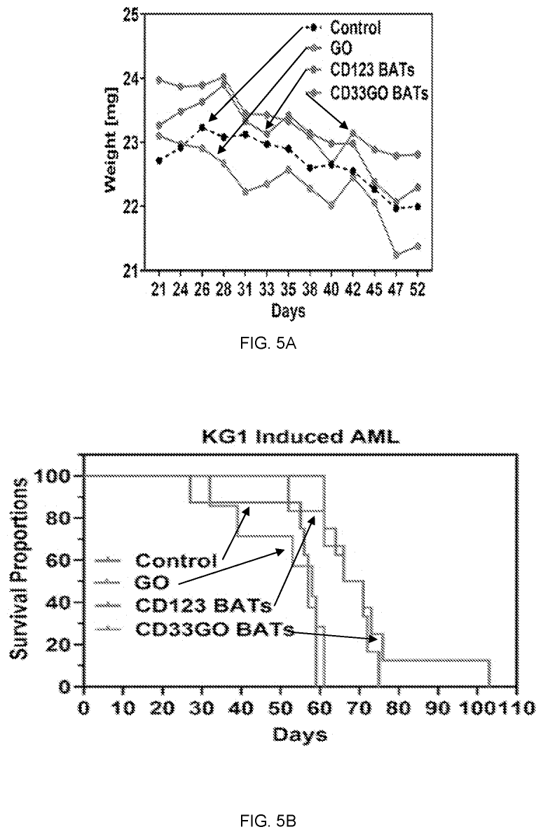

Results. BATs exhibited high levels of specific cytotoxicity directed at AML cell lines at low 1:1 or 1:2 E: T ratios and secreted Th₁ cytokines upon target engagement. Patient samples containing leukemic blasts and LSC treated with CD33GO BATs or CD123 BATs for 18 hours showed a significant reduction (50-100%: p<0.005) in blasts and 75-100% reduction in LSC (p<0).005) in most cases compared to unarmed ATC. The IV injections of CD33GO BATs or CD123 BATs (FIGS. 4 and 5) inhibited growth of leukemic cells in NSG mice compared to those who received GO alone (p<0.0006).

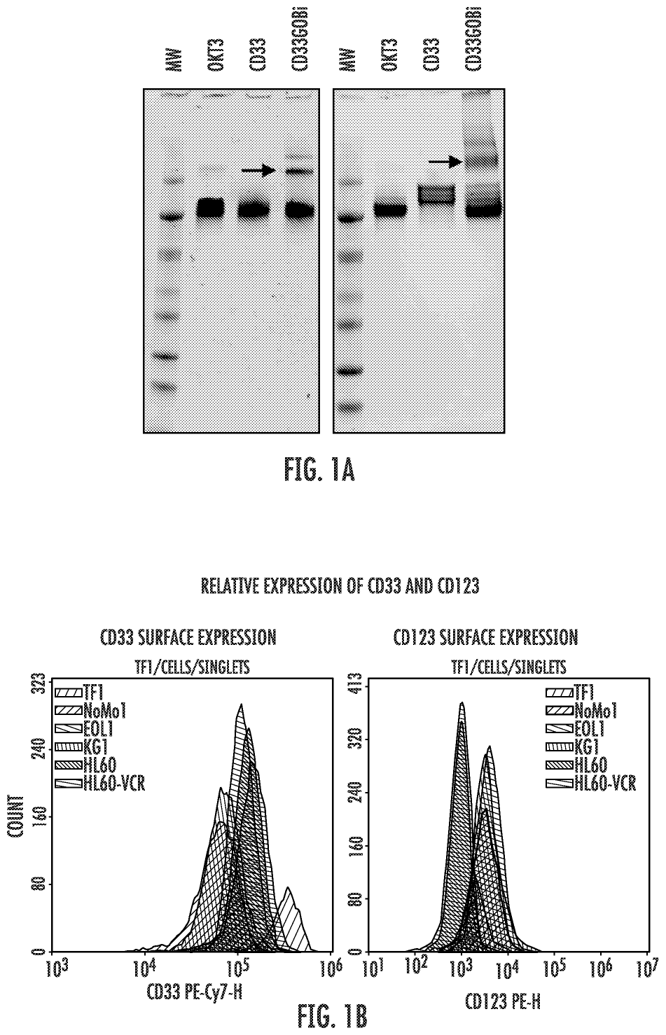

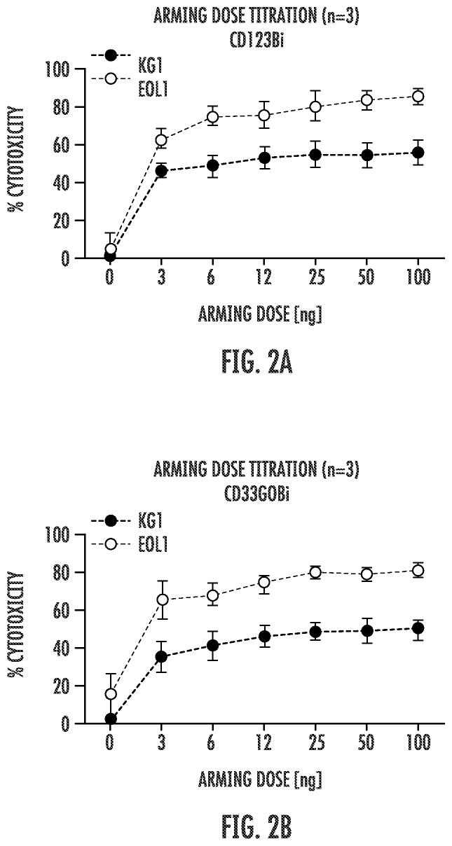

Conclusions. Both CD33GO BATs and CD123 BATs exhibited significantly high specific cytotoxicity against blast and LSC populations in patient samples. Vincristine and GO resistant cell line, primed with CD33GO BATs and CD123 BATs showed enhanced drug retention. This approach can provide a potent and non-toxic strategy to target AML blasts and LSCs and enhance chemo-responsiveness in older patients who are likely to develop recurrent disease (FIG. 1A-1B).

Flow Cytometry Based Cytotoxicity Assay. Employed was a quantitative flow cytometry-based cytotoxicity assay that is highly sensitive at low effector: target (E: T) in which the concentration of both effector T cells (BATs) and target cells (AML cell lines) is measured in fixed-volume aliquots at the time of initiation and after 18 hours (or more) of culture using an ACEA Biosciences (San Diego, CA) NovoCyte flow cytometer³³. Briefly, the target cells are fluorescently labeled with eFluor 450 dye (Invitrogen) and added to 24 or 96 well culture plates. ATC were added to target cells at the designated E: T ratios and gently mixed. At designated time points, fixed volumes of co-cultured cells were acquired to determine the number of live BATs and target cells as previously described³³. A forward/side scatter gate was drawn to capture the lymphocyte population followed by enumeration of live BATs (7-AAD negative/eFluor negative) and target cells (7-AAD negative/eFluor+). The number of live target cells is used to calculate the percent killing using the formula: 1-[Number of targets incubated with effector T cells at a given time point/Number of targets at the same time point cultured without effectors]×100%. The absolute number of live target cells is calculated using the formula: (Absolute starting number based on the absolute number in the sample)−(absolute number targets incubated with effector T cells at a given time point/(absolute number of targets cultured without effector).

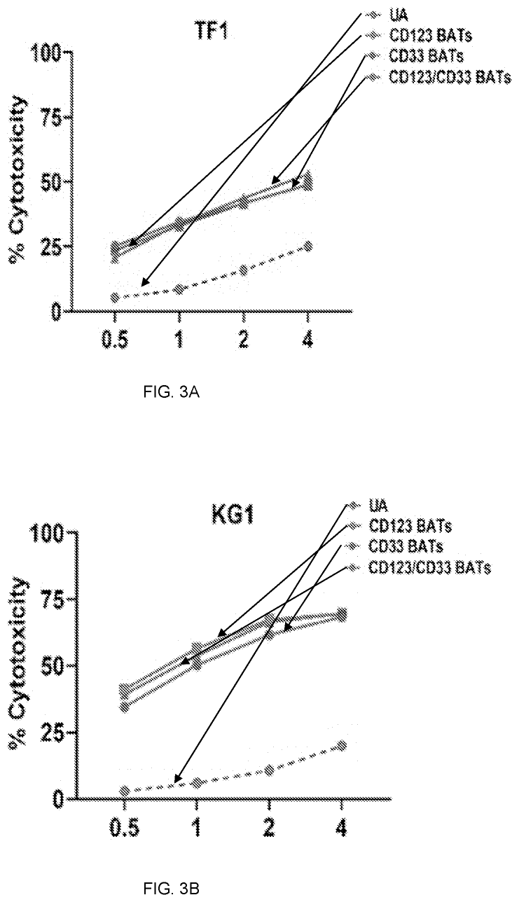

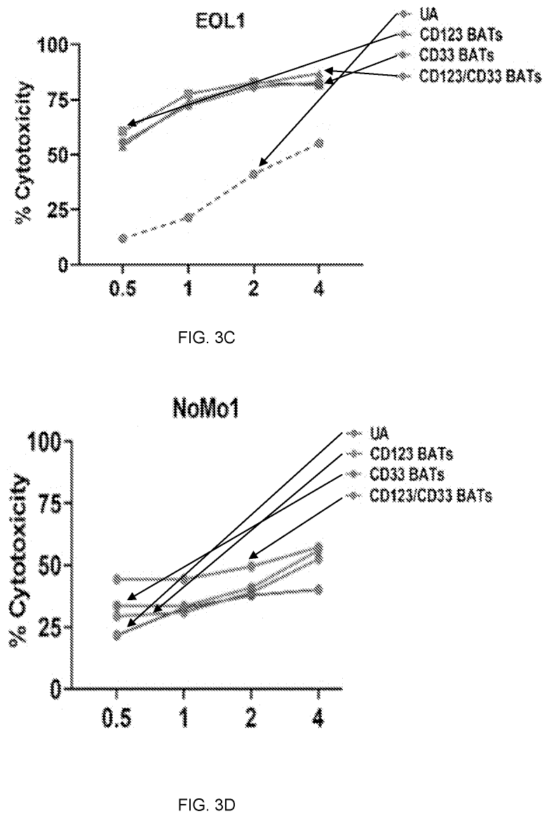

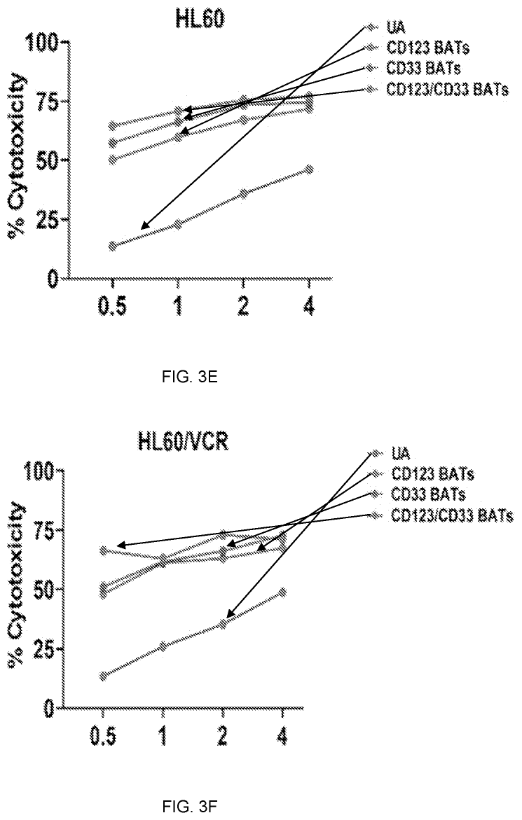

CD33GO BATs. ATC from 3 normal donors armed with CD33GOBi at 25 ng/106 cells exhibited significantly higher specific cytotoxicity at E: T ranging from 0.5:1-4:1 against TF1 ranging from 25-49% (p<0.03): KG1 from 41-70% (p<0.03): EOL1 from 61-82% (p<0).028): NoMo1 from 34-56% (p<0.05): HL60 from 57-75% (p<0.03) and HL60/VCR from 51-73% (p<0.03) than ATC which at the same E: Ts exhibited 5-55% cytotoxicity (FIGS. 3A-3F).

CD123 BATs. Specific cytotoxicity mediated by CD123 BATs was significantly higher at E: Ts ranging from 0.5:1-4:1 against KG1 from 34-68% (p<0.03), EOL1 from 56-83% (p<0).03), NoMo1 from 29-53% (p<0).8) and HL60 from 50-72% (p<0).028): however, TF1 (24-50%, p<0.057) and HL60/VCR (48-67%, p<0.057) showed no significant difference compared to ATC which at the same E: T exhibited 5-55% cytotoxicity (n=3).

CD33GO/CD123 BATs. Next, we asked whether mixing CD33GO BATs and CD123 BATs in equal amounts would exhibit enhanced specific cytotoxicity compared to the CD33GO BATs or CD123 BATs alone. Combined targeting with CD33GO BATs or CD123 BATs show significantly enhanced cytotoxicity against NoMo1 and HL60/VCR cell lines only at low E: T of 1:2 (p<0.0001 and p<0.002, respectively) while there was no added cytotoxic effect of combined targeting against TF1, KG1, EOL1 and HL60 cells lines (FIGS. 3A, 3C, 3E). At higher E: Ts of 4:1, specific cytotoxicity against all cell lines tested showed similar pattern as with single antigen targeting by CD33GO BATs or CD123 BATs.

To confirm the specificity of CD33GO BATs or CD123 BATs killing of AML cells, we used DAUDI cells, which do not express CD33 or CD123, at different E: T ratio of 0.5:1, 1:1, 2:1 and 4:1. Both, CD33GO BATs or CD123 BATs show low levels of LAK (lymphokine activated killer cells) like cytotoxicity mediated by ATC alone against DAUDI cells at all E: T (Data not shown) confirming specificity of CD33GO BATs or CD123 BATs for AML cell lines.

Induction of Cytotoxicity in Fresh PBMC by Arming with Anti-CD3 x anti-CD33 BiAb. Fresh PBMC were armed with anti-CD3 x anti-CD33GOBiAb or anti-CD3 x anti-CD123 BiAb leading to cytotoxicity directed at AML cell line EOL1 at an E: T of 2:1 using flow cytometry.

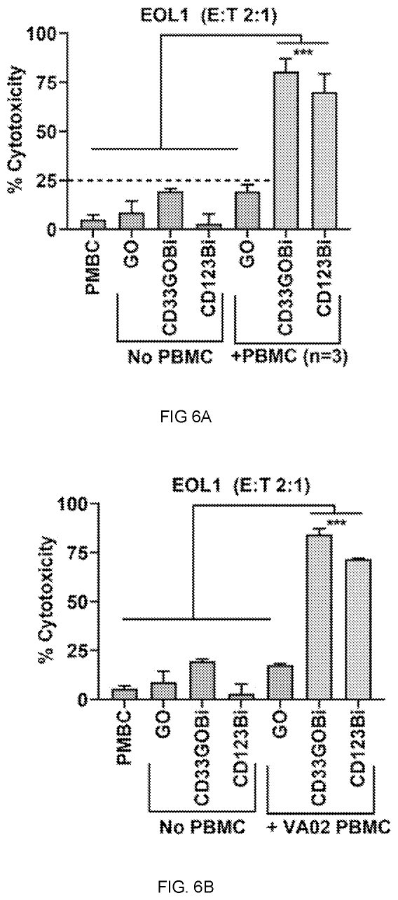

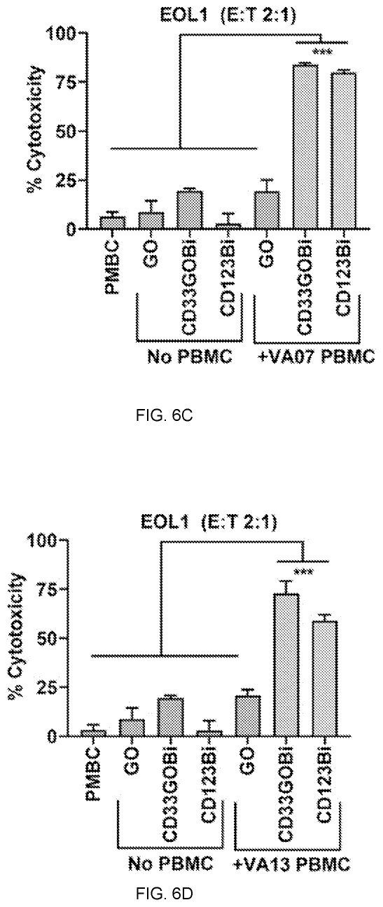

Low Levels of Cytotoxicity Mediated by GO Alone. CD33GOBi or CD123Bi Alone. Since IC50 dose of GO is 2.5 ng/ml for EOL1 cell line, we used the same concentration for overnight (18 h) cytotoxicity assay. Similarly, effective dose of 5 ng BiAb/well of CD33GOBi or CD123Bi against EOL1 cell line was used. Since the mechanism of action of GO is by internalization and cleavage to release calechiamicin independent of antibody dependent cellular cytotoxicity (ADCC), we carried out cytotoxicity assays for 18 hrs. to allow internalization of GO. Cytotoxicity with GO alone. CD33GOBi or CD123Bi alone was 8.7%. 19.5% and 2.9%, respectively, in the absence of PBMC (FIGS. 6A-6C). In the presence of PBMC (E/T 2:1) both CD33GOBi and CD123Bi showed significantly higher cytotoxicity of 80% (p<0.0001) and 70% (p<0.0001) respectively, compared PBMC alone. GO alone and BiAbs alone (FIG. 6D).

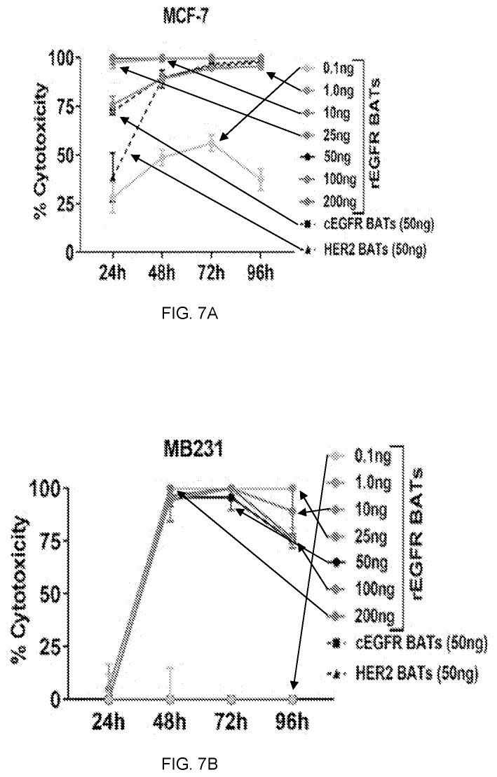

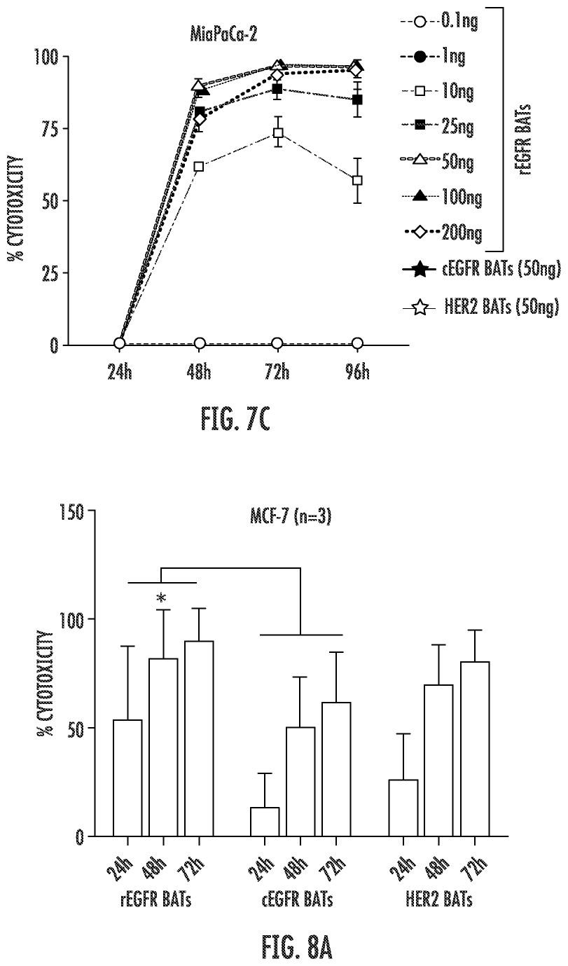

Titration of rEGFRBi on Activated T cells. In order to assess the ability of recombinant rEGFRBi to arm fresh immune effectors, the level of specific cytotoxicity mediated by rEGFRBi armed ATC directed at breast and pancreatic tumor cell lines was titrated (FIG. 7A-7B). The rEGFRBi at doses ranging from 10 to 200 ng per 106 ATC mediated high levels of specific cytotoxicity on all 3 cell lines (FIGS. 7A-7C). These data established the dose related activity of rEGFRBi for arming BATs. 20)

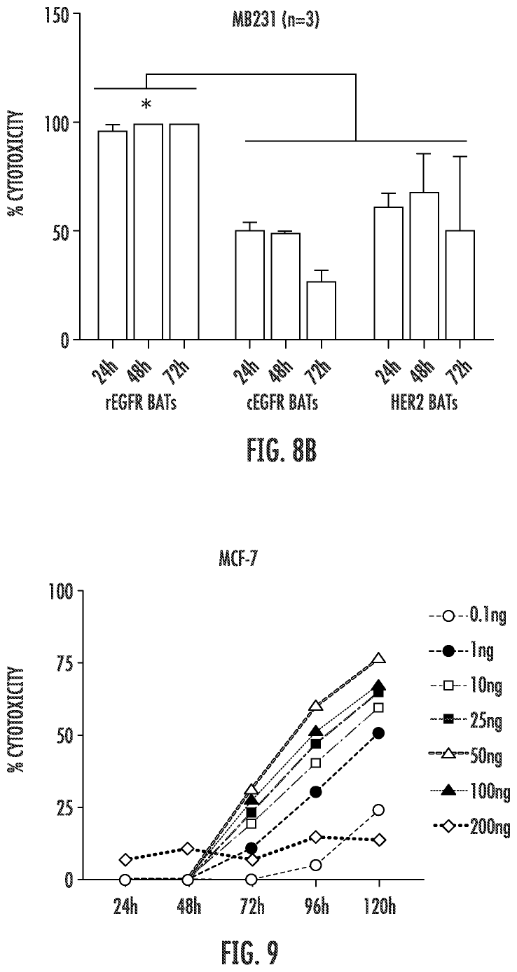

Comparison of cytotoxicity mediated by rEGFR BATs, cEGFR BATs, and HER2 BATs. ATC from three normal subjects were armed with 50 ng of BiAb/106 ATC and tested in real time cytotoxicity assay against MCF-7 (FIG. 8A) and MDA-MB-231 (FIG. 8B) at 24, 48, ad 72 hours to define the amounts and persistence of specific cytotoxicity. The rEGFR BATs clearly mediated higher levels of specific cytotoxicity than either 25 chemically heterconjugated HER2Bi or EGFRBi armed BATs.

Dose titration of rEGFRBi for arming Fresh PBMC. Arming fresh PBMC with various doses of rEGFRBi induce specific cytotoxicity (all corrected for background) directed at MCF-7 at all doses ranging from 0.1 to 200 ng/106 fresh PBMC. The rise in specific cytotoxicity begins around 72 hours after arming and continue to increase up to 120 30) hours (5 days) (FIG. 9).

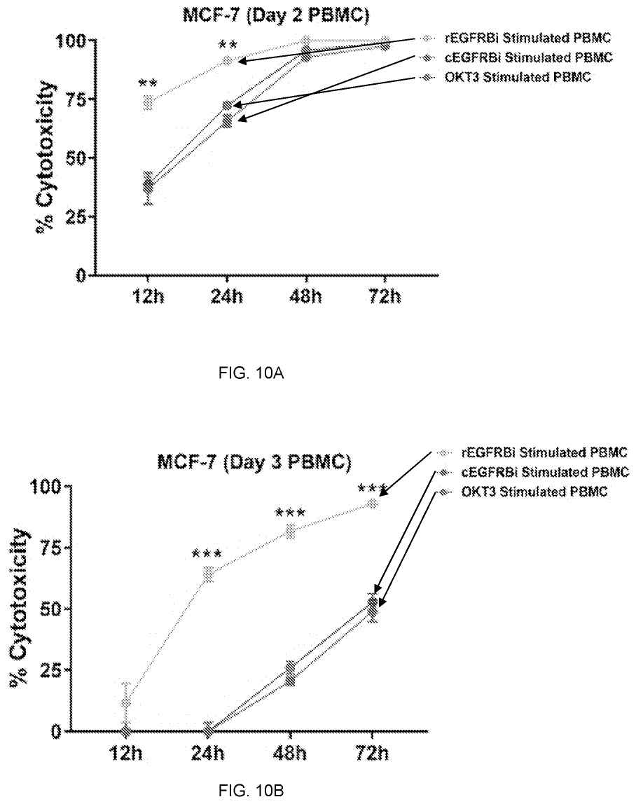

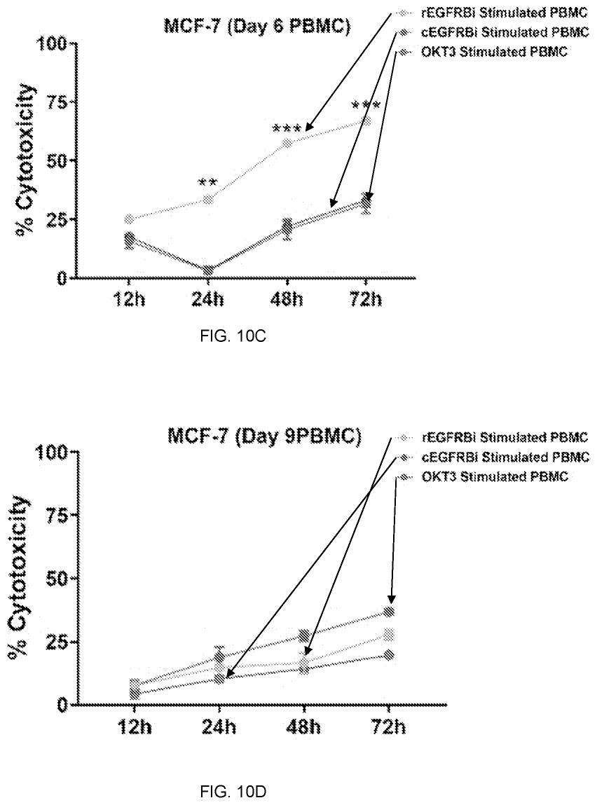

Cytotoxicity Mediated by Armed PBMC at different days of culture after Arming. Normal donor PBMC were activated by standard soluble OKT3 (20 ng/106 cells) and IL-2 (100IU/106 cells) or cEGFRBi (20 ng/106 cells)+IL-2 or rEGFRBi (20 ng/106 cells) +IL-2 at to determine whether OKT3 part of the BiAb in cEGFRBi and rEGFRBi can immune cells in PBMC. OKT3, cEGFRBi and rEGFRBi armed PBMC were tested for their cytotoxic activity on days 2, 3, 6 and 9 against MCF-7 cells without arming at an E: T of 1:1 on the day of harvest (FIGS. 10A-10D). The rEGFRBi PBMC showed significantly greater cytotoxicity ranging from (p<0.002-0.0001) on days. 2, 3 and 6 compared to OKT3 and cEGFRBi PBMCs (FIGS. 10A-10C). However, on day 9 and beyond cytotoxic activity by all three different cell populations were reduced drastically (FIG. 10D). Reduced cytotoxic activity by BiAb the BiAb activated PBMC is likely due to the reduced levels of BiAb on T cells that were armed and activated after multiple cell divisions by day 9 and beyond.

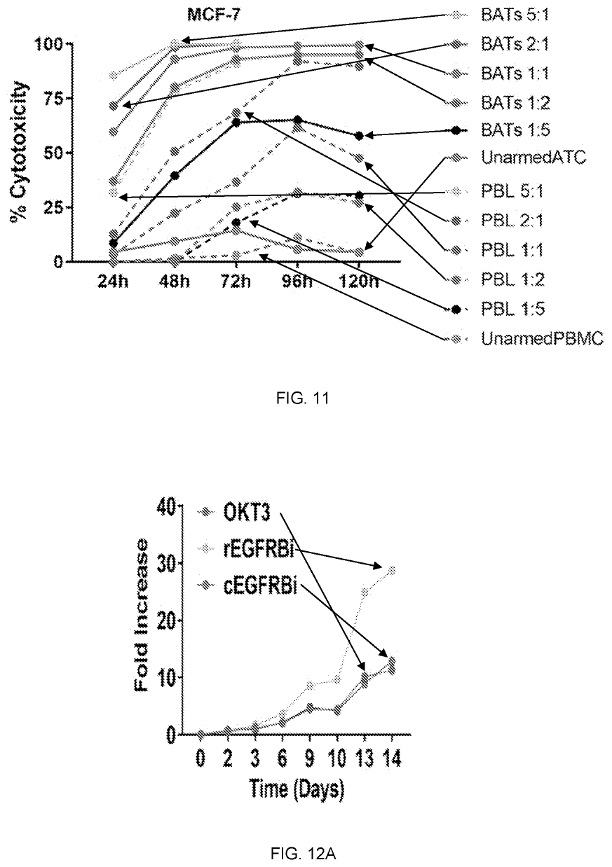

Comparison between BATs and Freshly Armed PBMC at varying E: T ratios. FIG. 11 shows the ability of armed PBMC compared to rEGFR BATs at low E: T to mediate cytotoxicity between 24 and 120 hours. The data show that specific cytotoxicity emerges over 24-96 hours in freshly armed PBMC at low E: T.

Proliferation Induced by arming PBMC with both chemical or recombinant bispecific antibody targeting EGFR. A unique property of fresh PBMC with anti-CD3 based-bispecific antibodies is that arming process induces proliferation of the T cells in the PBMC (FIG. 12). These data show that chemical heteroconjugated anti-CD3 x anti-EGFR BiAb and the rEGFRBi (anti-CD3 x anti-EGFR) are both able to induce proliferation of PBMC up to 14 days. The proliferation induced by soluble OKT3 (20 ng/ml) is shown for comparison. FIG. 12A shows proliferation on a linear scale and FIG. 12B show proliferation 20 on a log scale. These data show that BiAb activation of the T cells in PBMC induces proliferation as well as specific cytotoxicity.

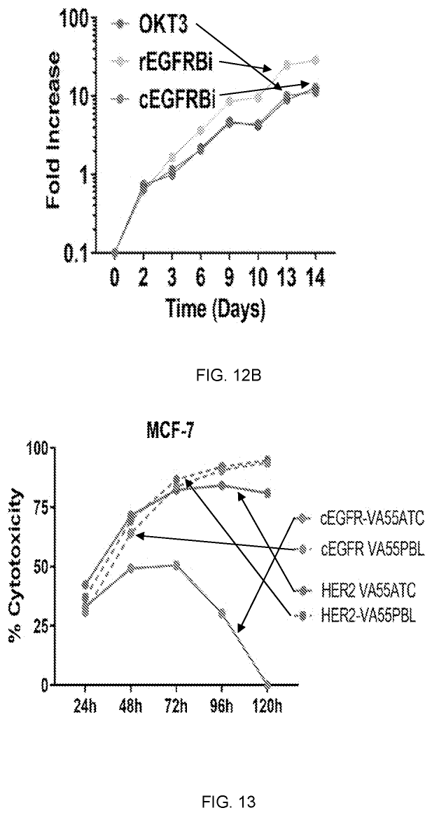

Comparison of BiAb induced Cytotoxicity by ATC and Fresh PBMC Directed at MCF-7. HER2Bi armed PBMC and cEGFRBi armed PBMC mediated high levels of specific cytotoxicity beginning 24 hours after arming lasting up to 12 hours of culture (FIG. 13).

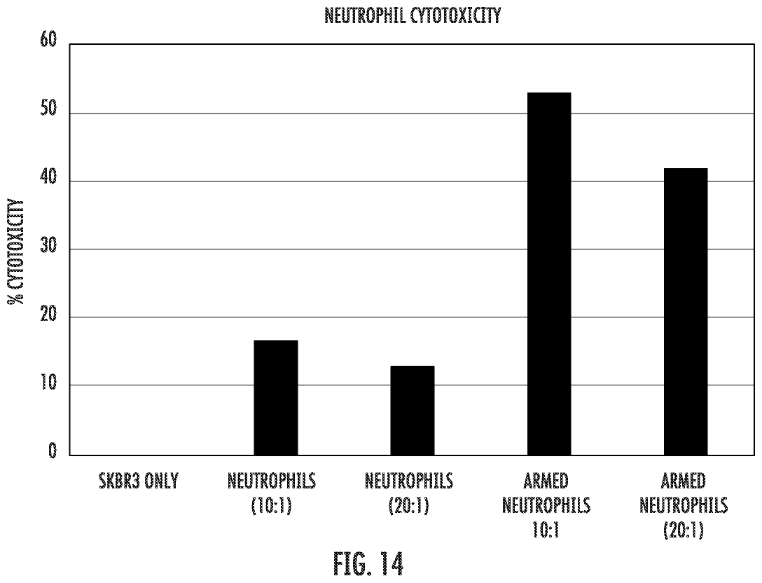

Arming of Neutrophils with anti-CD89 x anti-Her2 Bispecific Antibody. In order to determine whether the Fc-IgA receptor on neutrophils can be used to redirect neutrophil mediated cytotoxicity to kill solid tumors, anti-CD89 x anti-Her2 bispecific antibody was produced by chemical heteroconjugation. Neutrophils were isolated with MACSxpress neutrophil isolation and MACSxpress using erythrocyte depletion kits (Miltenyi Biotec. Inc., Auburn. CA). The neutrophils were resuspended in RPMI 1640 (without phenol red) supplemented with fetal bovine serum (FBS) and glutamine (0.3 mg/ml). The neutrophils were incubated at 37° C. with 5% CO₂. SK-BR-3 targets were plated in the RTCA wells and neutrophils were added to the triple wells at E: T of 10:1 and 20:1. Arming with anti-CD89 (clone number MIP7c) x anti-HER2 (Herceptin) Biab was performed at an arming dose of 500 ng/10⁶. There were 5 other experiments performed in a similar manner in which fresh PMNs armed with anti-CD89 x anti-HER2 bispecific antibody enhanced neutrophil mediated killing of Sk-BR-3 breast cancer cell line at E: T ratios in the same range. (FIG. 14).

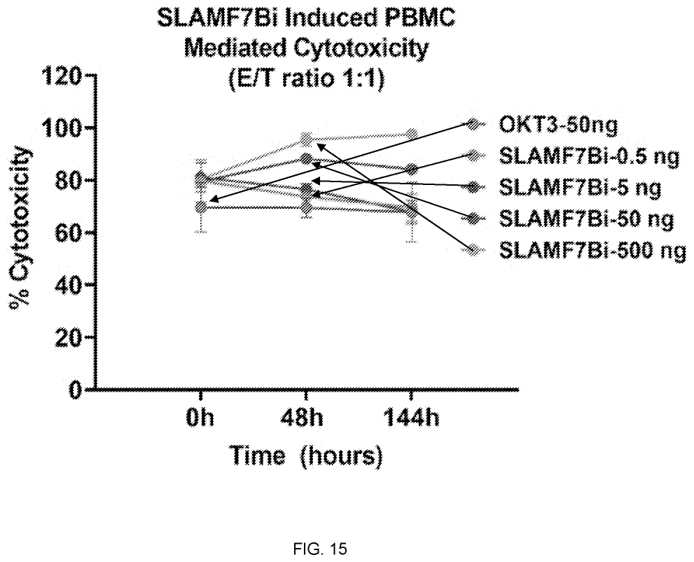

Kinetics of Cytotoxicity at Very Low to Very High Activation Doses of SLAMF7Bi FPBMC over time. Fresh PBMC from 2 normal donors were activated at various doses (0.5, 5.0, 50 and 500 ng/106 FPBMC) of SLAMF7Bi (anti-CD3 x anti SLAMF7 (anti-CS-1)) compared standard 50 ng-OKT3 induced activation. Following activation PBMC were tested for their cytotoxic activity at 0, 48 and 144 hours of culture against MM. IS multiple myeloma cell line at an E: T of 1:1. All four activation doses of SLAMF7Bi showed remarkable cytotoxicity ranging from 77-80% immediately after activation, at 48 hours cytotoxicity ranged 85-93% and remained high (80-95%) even after 6 days in culture against MM. IS cell line at the low E: T of 1:1 (FIG. 15).

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
1. Short N J, Rytting M E, Cortes J E. Acute myeloid leukaemia. Lancet 2018:392:593-606.
2. DiNardo C D, Perl A E. Advances in patient care through increasingly individualized therapy. Nat Rev Clin Oncol 2019:16:73-4.
3. Papaemmanuil E, Gerstung M, Bullinger L, et al. Genomic Classification and Prognosis in Acute Myeloid Leukemia. N Engl J Med 2016; 374:2209-21.
4. Kantarjian H. Acute myeloid leukemia—major progress over four decades and glimpses into the future. Am J Hematol 2016:91:131-45.
5. Kadia T M, Ravandi F, Cortes J, Kantarjian H. Toward Individualized Therapy in Acute Myeloid Leukemia: A Contemporary Review. JAMA Oncol 2015:1:820-8.
6. Kadia T M, Ravandi F, O'Brien S, Cortes J, Kantarjian H M. Progress in acute myeloid leukemia. Clin Lymphoma Myeloma Leuk 2015:15:139-51.
7. Short N J, Konopleva M, Kadia T M, et al. Advances in the Treatment of Acute Myeloid Leukemia: New Drugs and New Challenges. Cancer Discov 2020:10:506-25.
8. Loke J, Malladi R, Moss P, Craddock C. The role of allogeneic stem cell transplantation in the management of acute myeloid leukaemia: a triumph of hope and experience. Br J Haematol 2020:188:129-46.
9. van Balen P, van Bergen C A M, van Luxemburg-Heijs S A P, et al. CD4 Donor Lymphocyte Infusion Can Cause Conversion of Chimerism Without GVHD by Inducing Immune Responses Targeting Minor Histocompatibility Antigens in HLA Class II. Front Immunol 2018:9:3016.
10. van Bergen C A, van Luxemburg-Heijs S A, de Wreede L C, et al. Selective graft-versus-leukemia depends on magnitude and diversity of the alloreactive T cell response. J Clin Invest 2017:127:517-29.
11. Battipaglia G, Labopin M, Kroger N, et al. Posttransplant cyclophosphamide vs antithymocyte globulin in HLA-mismatched unrelated donor transplantation. Blood 2019:134:892-9.
12. Ayuk F, Beelen D W, Bornhauser M, et al. Relative Impact of HLA Matching and Non-HLA Donor Characteristics on Outcomes of Allogeneic Stem Cell Transplantation for Acute Myeloid Leukemia and Myelodysplastic Syndrome. Biol Blood Marrow Transplant 2018:24:2558-67.
13. Mathew N R, Baumgartner F, Braun L, et al. Sorafenib promotes graft-versus-leukemia activity in mice and humans through IL-15 production in FLT3-ITD-mutant leukemia cells. Nat Med 2018:24:282-91.
14. Ayuk F, Zabelina T, Wortmann F, et al. Donor choice according to age for alloSCT for AML in complete remission. Bone Marrow Transplant 2013:48:1028-32.
15. Hauswirth A W, Florian S, Printz D, et al. Expression of the target receptor CD33 in CD34+/CD38−/CD123+ AML stem cells. Eur J Clin Invest 2007:37:73-82.
16. Jordan C T, Upchurch D, Szilvassy S J, et al. The interleukin-3 receptor alpha chain is a unique marker for human acute myelogenous leukemia stem cells. Leukemia 2000:14:1777-84.
17. Han Y C, Kahler J, Piche-Nicholas N, et al. Development of Highly Optimized Antibody-Drug Conjugates against CD33 and CD123 for Acute Myeloid Leukemia. Clin Cancer Res 2021:27:622-31.
18. Cortes J E, de Lima M, Dombret H, et al. Prevention, recognition, and management of adverse events associated with gemtuzumab ozogamicin use in acute myeloid leukemia. J Hematol Oncol 2020:13:137.
19. Anderson D R, Grillo-Lopez A, Varns C, Chambers K S, Hanna N. Targeted anti-cancer therapy using rituximab, a chimaeric anti-CD20 antibody (IDEC-C2B8) iin the treatment of non-Hodgkin's B-cell lymphoma. BiochemSocTrans 1997:25:705-8.
20. Horwitz S M, Negrin R S, Blume K G, et al. Rituximab as adjuvant to high-dose therapy and autologous hematopoietic cell transplantation for aggressive non-Hodgkin lymphoma. Blood 2004:103:777-83.
21. Kamezaki K, Kikushige Y, Numata A, et al. Rituximab does not compromise the mobilization and engraftment of autologous peripheral blood stem cells in diffuse-large B-cell lymphoma. Bone Marrow Transplant 2007:39:523-7.
22. Maloney D G, Grillo-López AJ, White C A, et al. IDEC-C2B8 (Rituximab) anti-CD20 monoclonal antibody therapy in patients with relapsed low-grade non-Hodgkin's lymphoma. Blood 1997:90:2188-95.
23. Coiffier B, Thieblemont C, Van Den N E, et al. Long-term outcome of patients in the LNH-98.5 trial, the first randomized study comparing rituximab-CHOP to standard CHOP chemotherapy in DLBCL patients: a study by the Groupe d'Etudes des Lymphomes de l'Adulte. Blood 2010:116:2040-5.
24. Bosly A, Coiffier B, Gisselbrecht C, et al. Bone marrow transplantation prolongs survival after relapse in aggressive-lymphoma patients treated with the LNH-84 regimen. JClinOncol 1992:10:1615-23.
25. Cardoso F, Di L A, Lohrisch C, Bernard C, Ferreira F, Piccart M J. Second and subsequent lines of chemotherapy for metastatic breast cancer: what did we learn in the last two decades? AnnOncol 2002:13:197-207.
26. Li C H, Karantza V, Aktan G, Lala M. Current treatment landscape for patients with locally recurrent inoperable or metastatic triple-negative breast cancer: a systematic literature review. Breast Cancer Res 2019:21:143.
27. Dufresne A, Pivot X, Tournigand C, et al. Impact of chemotherapy beyond the first line in patients with metastatic breast cancer. Breast Cancer ResTreat 2007.
28. O'shaughnessy J, Miles D, Vukelja S, et al. Superior survival with capecitabine plus docetaxel combination therapy in anthracycline-pretreated patients with advanced breast cancer: phase III trial results. JClinOncol 2002:20:2812-23.
29. Keller A M, Mennel R G, Georgoulias V A, et al. Randomized phase III trial of pegylated liposomal doxorubicin versus vinorelbine or mitomycin C plus vinblastine in women with taxane-refractory advanced breast cancer. JClinOncol 2004:22:3893-901.
30. Martin M, Ruiz A, Munoz M, et al. Gemcitabine plus vinorelbine versus vinorelbine monotherapy in patients with metastatic breast cancer previously treated with anthracyclines and taxanes: final results of the phase III Spanish Breast Cancer Research Group (GEICAM)trial. Lancet Oncol 2007:8:219-25.
31. Pivot X, Im S A, Guo M, Marme F. Subgroup analysis of patients with HER2-negative metastatic breast cancer in the second-line setting from a phase 3, open-label, randomized study of eribulin mesilate versus capecitabine. Breast Cancer 2018:25:370-4.
32. Perez E A, Awada A, O'Shaughnessy J, et al. Etirinotecan pegol (NKTR-102) versus treatment of physician's choice in women with advanced breast cancer previously treated with an anthracycline, a taxane, and capecitabine (BEACON): a randomised, open-label, multicentre, phase 3 trial. Lancet Oncol 2015:16:1556-68.
33. Twelves C, Cortes J, Vahdat L, et al. Efficacy of eribulin in women with metastatic breast cancer: a pooled analysis of two phase 3 studies. Breast Cancer Res Treat 2014:148:553-61.
34. Brufsky A M, Hurvitz S, Perez E, et al. RIBBON-2: a randomized, double-blind, placebo-controlled, phase III trial evaluating the efficacy and safety of bevacizumab in combination with chemotherapy for second-line treatment of human epidermal growth factor receptor 2-negative metastatic breast cancer. J Clin Oncol 2011:29:4286-93.
35. Society A C. Cancer Facts and Figures 2019. 2019.
36. Siegel R L, Miller K D, Jemal A. Cancer statistics, 2018. CA Cancer J Clin 2018:68:7-30.
37. Von Hoff D D, Ervin T, Arena F P, et al. Increased survival in pancreatic cancer with nab-paclitaxel plus gemcitabine. N Engl J Med 2013:369:1691-703.
38. Conroy T, Desseigne F, Ychou M, et al. FOLFIRINOX versus Gemcitabine for Metastatic Pancreatic Cancer. New England Journal of Medicine 2011:364: 1817-25.
39. Rahma O E, Duffy A, Liewehr D J, Steinberg S M, Greten T F. Second-line treatment in advanced pancreatic cancer: a comprehensive analysis of published clinical trials. Annals of oncology: official journal of the European Society for Medical Oncology/ESMO 2013:24:1972-9.
40. Ostrom Q T, Patil N, Cioffic G, Waite K, Kruchko C, Barnhotz-Sloan J S. CBTRUS Statistical Report: Primary Brain and Other Central Nervous System Tumors Diagnosed in the United States in 2013-2017. Neuro Oncol 2020:22.
41. Ostrom Q T, Bauchet L, Davis F G, et al. The epidemiology of glioma in adults: a “state of the science” review. Neuro Oncol 2014:16:896-913.
42. Stupp R, Hegi M E, Mason W P, et al. Effects of radiotherapy with concomitant and adjuvant temozolomide versus radiotherapy alone on survival in glioblastoma in a randomised phase III study: 5-year analysis of the EORTC-NCIC trial. Lancet Oncol 2009:10:459-66.
43. Honsik C J, Jung G, Reisfeld R A. Lymphokine-activated killer cells targeted by monoclonal antibodies to the disialogangliosides GD2 and GD3 specifically lyse human tumor cells of neuroectodermal origin. ProcNatlAcadSciUSA 1986:83:7893-7.
44. Matthay K K, George R E, Yu A L. Promising therapeutic targets in neuroblastoma. Clin Cancer Res 2012:18:2740-53.
45. Tarek N, Le Luduec J B, Gallagher M M, et al. Unlicensed N K cells target neuroblastoma following anti-GD2 antibody treatment. J Clin Invest 2012:122:3260-70.
46. Cheung I Y, Hsu K, Cheung N K. Activation of peripheral-blood granulocytes is strongly correlated with patient outcome after immunotherapy with anti-GD2 monoclonal antibody and granulocyte-macrophage colony-stimulating factor. J Clin Oncol 2012:30:426-32.
47. Navid F, Santana V M, Barfield R C. Anti-GD2 antibody therapy for GD2-expressing tumors. CurrCancer Drug Targets 2010:10:200-9.
48. Yu A L, Gilman A L, Ozkaynak M F, et al. Anti-GD2 antibody with GM-CSF, interleukin-2, and isotretinoin for neuroblastoma. N Engl J Med 2010:363:1324-34.
49. Simon T, Hero B, Faldum A, et al. Long term outcome of high-risk neuroblastoma patients after immunotherapy with antibody ch14.18 or oral metronomic chemotherapy. BMCCancer 2011:11:21.
50. PCT International Publication No. WO 2020/176880, Published Sep. 3, 2020.
51. PCT International Publication No. WO 2020/198678, Published Oct. 1, 2020.
52. PCT International Publication No. WO 2021/026074, Published Feb. 11, 2021.
53. Published U.S. Patent Application No. US2018/0243341A1 to June et al., published Aug. 30, 2018
54. Published U.S. Patent Application No. US20180282693A1 to June et al., published Oct. 4, 2018
55. Van Meir E G, Hadjipanayis C G, Norden A D, Shu H K, Wen P Y, Olson J J. Exciting new advances in neuro-oncology: the avenue to a cure for malignant glioma. CA Cancer J Clin. 2010 May-Jun;60 (3): 166-93.
56. Vaishampayan U N, Thakur A, et al. Phase II trial of Pembrolizumab and Anti-CD3 x Anti-HER2 Bispecific Antibody Armed Activated T Cells in Metastatic Castrate Resistant Prostate Cancer. Clin Cancer Res. 2022 Oct. 18: CCR-22-1601.
57. Wu L, Seung E, et al. Trispecific antibodies enhance the therapeutic efficacy of tumor-directed T cells through T cell receptor co-stimulation. Nat Cancer. 2020 January; 1 (1): 86-98.
58. Au K M, Park S I, Wang A Z. Trispecific natural killer cell nanoengagers for targeted chemoimmunotherapy. Sci Adv. 2020 Jul. 3;6 (27).
59. Yun H D, Felices M, et al. Trispecific killer engager CD16xIL15xCD33 potently induces NK cell activation and cytotoxicity against neoplastic mast cells. Blood Adv. 2018 Jul. 10;2 (13): 1580-1584.
60 Schneider D, Xiong Y, et al. Trispecific CD19-CD20-CD22-targeting duoCAR-T cells eliminate antigen-heterogeneous B cell tumors in preclinical models. Sci Transl Med. 2021 Mar. 24;13 (586)

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

What is claimed is:

1. A method for treatment of a patient suffering from a cancer, an autoimmune disease, an infection, a neurodegenerative disease or any combination thereof, said method comprising the steps of:
(a) providing fresh immune effector cells (IECs) armed with one or more multispecific antibodies, wherein the multispecific antibodies are bound to the fresh IECs; and
(b) administering an effective amount of a composition of cells to the patient, wherein the composition of cells comprises the multispecific antibody bound fresh IECs.

2. The method of claim 1, wherein the fresh IECs comprises a cell selected from the group consisting of peripheral blood mononuclear cells (PBMC), antigen-presenting cells, T cells (optionally fresh BiAb armed T cells (FBATs)), NK cells, monocytes, polymorphonuclear neutrophils (PMNs), and any combination thereof.

3. The method of claim 1 or claim 2, wherein providing the fresh IECs comprises isolating fresh IECs from the patient.

4. The method of any one of claims 1 to 3, comprising arming fresh IECs with one or more multispecific antibodies, wherein the multispecific antibodies are bound to the fresh IECs.

5. The method of any one of claims 1 to 4, wherein the IECs are autologous or allogenic to the patient.

6. The method of any one of claims 1 to 5, wherein the patient is given IL-2, IL-7, IL-12, IL-15, and/or GM-CSF and/or a checkpoint inhibitor.

7. The method of any one of claims 1 to 6, wherein the composition of cells comprises augmenting cytokines IL-2, IL-7, IL-15, IL-12, and/or GM-CSF and/or a checkpoint inhibitor.

8. The method of any one of claims 2 to 7, further comprising co-administering a composition of antigen-presenting cells (APC), and said FBATs armed with multispecific antibody: wherein the APC and the FBATs are autologous to the patient, and the patient is given IL-2, IL-7, IL-15, and/or GM-CSF and/or a check point inhibitor.

9. The method according to any one of claims 1 to 8, wherein the multispecific antibody comprises two monoclonal antibodies.

10. The method according to any one of claims 1 to 9, wherein the multispecific antibody comprises a specificity for a tumor antigen.

11. The method according to any one of claims 1 to 10, wherein the multispecific antibody comprises a specificity for a T cell receptor complex.

12. The method according to any one of claims 1 to 11, wherein the multispecific antibody comprises specificities for a tumor antigen and a T cell receptor complex.

13. The method according to any one of claims 1 to 12, wherein the multispecific antibody comprises monoclonal antibodies that are chemically heteroconjugated to form a bispecific antibody.

14. The method according to any one of claims 1 to 13, wherein the multispecific antibody comprises an antibody construct of a plurality of specificities and a cytokine/chemokine receptor.

15. The method according to any one of claims 1 to 14, wherein the multispecific antibody comprises an antibody construct having at least three specificities, wherein one of the at least three specificities comprises a T cell receptor complex.

16. The method according to any one of claims 1 to 15, wherein the multispecific antibody comprises an antibody construct having at least three specificities and contains an antibody drug conjugate.

17. The method according to any one of claims 1 to 16, wherein the multispecific antibody comprises an antibody construct having at least four specificities, and contains a cytokine/chemokine receptor.

18. The method according to any one of claims 1 to 17, wherein an anti T cell receptor monoclonal antibody component of the multispecific antibody is directed against a CD3 of the T cell receptor complex.

19. The method according to any one of claims 1 to 18, wherein the patient is immunosuppressed.

20. The method according to any one of claims 1 to 19, wherein the patient is susceptible to, or suffering from a disease associated with abnormal cellular proliferation or growth.

21. The method according to any one of claims 1 to 20, wherein the patient is susceptible to, or suffering from a disease associated with an autoimmune reaction.

22. The method according to any one of claims 1 to 21, wherein said method further comprises freezing the composition of cells prior to administering the composition of cells to the patient in need of therapy.

23. The method of any one of claims 1 to 22, wherein said method further comprises thawing the composition of cells prior to administering the composition of cells to the patient in need of therapy.

24. The method of any one of claims 1 to 23, wherein the IECs are armed with a dose of one or more multispecific antibodies of about 0.1 ng per million fresh IECs to about 1000 ng per million fresh IECs.

25. The method of any one of claims 1 to 24, wherein a dose administered to the patient is optimized for each individual patient by titrating fresh and thawed aliquot of the composition of cells to achieve a percent specific cytotoxicity level at an effector to target (E: T) ratio from about 10:1 to at least about 10% against a tumor target.

26. The method of any one of claims 1 to 25, wherein the dose administered to the patient is at least about 0.5×10⁶ the composition of cells per kilogram body weight of the patient with a dose schedule that can range from at least 1 administration to 24 administrations.

27. The method of any one of claims 1 to 26, wherein the dose administered to the patient is administered ranging from once a week to 1 once per month up to a year.

28. The method of any one of claims 1 to 27, further comprising infusing intravenously or injecting into a tumor arterial supply or tumor site said IECs armed with multispecific antibody: wherein said IECs armed with bispecific antibody are derived from an autologous donor or from an allogeneic donor.

29. The method of any one of claims 1 to 28, wherein the composition of cells administered into a patient is free of soluble multispecific antibody.

30. The method of any one of claims 1-29, wherein the cancer is selected from the group consisting of prostate cancer, breast cancer, leukemia, colon cancer, brain cancer, lung cancer, ovarian cancer, osteosarcoma, and neck cancer.

31. A method for treatment of a patient suffering from cancer, said method comprising the steps of: (a) isolating a sample of peripheral blood mononuclear cells, comprising T cells, from a patient suffering from cancer: (b) arming one or more of said T cells with multispecific antibody: (c) arming of said T cells with multispecific antibody capable of binding to the T cell receptor complex of a T cell, and to tumor-associated antigens on a tumor cell, under conditions wherein: (i) said bispecific antibody binds to said T cells, tumor cells, and Fc receptor positive cells, (ii) said antibody binds to the tumor target and said antibody binding to said tumor target activates said T cells, (iii) said antibody redirects said T cells and Fc-receptor positive cells to said tumor cells, (iv) said bispecific antibody activated T cells destroy said tumor cells; and, (d) reinfusing, into the patient, a composition of autologous cells comprising said bispecific antibody armed activated T cells (FBAT) as treatment of the patient wherein: (i) contacting T cells specific for different epitopes on the tumor cell with the autologous cells and including proliferation of the T cells specific for different epitopes on the tumor cells, or targeting and contacting multiple target cells with the armed T cell to which the BiAb remains bound, and killing said multiple target cells.

32. A method of treatment, comprising: (a) providing a formulation comprising: (i) a pharmaceutically acceptable excipient; and (ii) PBMC having bound thereto a bispecific antibody with a binding specificity for a T-cell antigen, selected from the group consisting of CD28 and CD3, and a binding specificity for a CD33, CD123, CD20, SLAMF7, BCMA, other liquid tumor antigens, HER2, EGFR, GD2, CEA, PSMA, PSA, VEGF, other solid tumor antigen present on a surface of a cancer cell, wherein the bispecific antibody has a binding specificity of about 10-8 moles/liter or higher: (b) infusing said formulation into a human patient suffering from cancer: (c) binding the bispecific antibodies to cancer cells in the patient: (d) contacting the cancer cells in the patient with the PBMCs and lysing the cancer cells in the patient: (e) creating a T cell memory in the patient's endogenous T cells: and (f) contacting cancer cells in the patient with the patient's endogenous T cells and lysing said cancer cells.

33. A method of preparing a composition of cells comprising:
(a) isolating fresh immune effector cells (IECs) from a patient; and
(b) arming the fresh IECs with one or more multispecific antibodies, wherein the multispecific antibodies are bound to the fresh IECs without culture.

34. The method of claim 33, further comprises freezing the composition of cells.

35. The method of claim 33 or 34, further comprising (c) infusing into a subject armed IECs without culture or cryopreservation.

36. A composition produced by a method of any one of claims 32 to 35.

37. A composition comprising fresh immune effector cells (IECs) armed with one or more multispecific antibodies, wherein the one or more multispecific antibodies are bound to the fresh IECs.

38. A composition for use in treating a patient suffering from a cancer, an autoimmune disease, a neurodegenerative disorder, an infection and/or any combination thereof, wherein the composition comprises fresh immune effector cells (IECs) armed with one or more multispecific antibodies, wherein the multispecific antibodies are bound to the fresh IECs.

39. Use of a composition comprising fresh immune effector cells (IECs) armed with one or more multispecific antibodies, wherein the one or more multispecific antibodies are bound to the fresh IECs for the manufacture of a medicament for treating a cancer, an autoimmune disease, a neurodegenerative disorder, an infection and/or combination thereof.

40. The composition or use according to any one of claims 37-39, wherein the fresh IECs comprises a cell selected from the group consisting of peripheral blood mononuclear cells (PBMC), an antigen-presenting cells, T cell (optionally fresh BiAb armed T cells (FBATs)), NK cells, monocytes, polymorphonuclear neutrophils (PMNs), and any combination thereof.

41. The composition or use according to any one of claims 37-40, wherein the fresh IECs are isolated from a patient, optionally a patient to be treated.

42. The composition or use according to any one of claims 37-41, wherein the IECs are autologous or allogenic to the patient.

43. The composition or use according to one of claims 37-42, wherein the composition comprises augmenting cytokines IL-2, IL-7, IL-15, IL-12, and/or GM-CSF and/or a checkpoint inhibitor.

44. The composition or use according to one of claims 37-42, wherein the multispecific antibody comprises two monoclonal antibodies.

45. The composition or use according to one of claims 37-44, wherein the multispecific antibody comprises a specificity for a tumor antigen.

46. The composition or use according to one of claims 37-45, wherein the multispecific antibody comprises a specificity for a T cell receptor complex.

47. The composition or use according to one of claims 37-46, wherein the multispecific antibody comprises specificities for a tumor antigen and a T cell receptor complex.

48. The composition or use according to one of claims 37-47, wherein the multispecific antibody comprises monoclonal antibodies that are chemically heteroconjugated to form a bispecific antibody.

49. The composition or use according to one of claims 37-48, wherein the multispecific antibody comprises an antibody construct of a plurality of specificities and a cytokine/chemokine receptor.

50. The composition or use according to one of claims 37-49, wherein the multispecific antibody comprises an antibody construct having at least three specificities wherein one of the at least three specificities comprises a T cell receptor complex.

51. The composition or use according to one of claims 37-50, wherein the multispecific antibody comprises an antibody construct having at least three specificities and contains an antibody drug conjugate.

52. The composition or use according to one of claims 37-51, wherein the multispecific antibody comprises an antibody construct having at least four specificities, wherein one the at least four specificities and contains a cytokine/chemokine receptor.

53. The composition or use according to one of claims 37-52, wherein an anti T cell receptor monoclonal antibody component of the multispecific antibody is directed against a CD3 of the T cell receptor complex.

54. The composition or use according to one of claims 37-53, wherein said composition is frozen, optionally wherein the composition is thawed prior to administering the composition of cells to a patient in need of therapy.

55. The composition or use according to one of claims 37-54, wherein the IECs are armed with a dose of one or more multispecific antibodies of about 0.1 ng per million fresh IECs to about 1000 ng per million fresh IECs.

56. The composition or use according to one of claims 37-55, wherein a dose administered to the patient is optimized for each individual patient by titrating fresh and thawed aliquot of the composition of cells to achieve a percent specific cytotoxicity level at an effector to target (E: T) ratio from about 10:1 to at least about 10% against a tumor target.

57. The composition or use according to one of claims 38-56, wherein the patient is immunosuppressed.

58. The composition or use according to one of claims 38-57, wherein the patient is susceptible to, or suffering from a disease associated with abnormal cellular proliferation or growth.

59. The composition or use according to one of claims 38-58, wherein the patient is susceptible to, or suffering from a disease associated with an autoimmune reaction.

60. The method, composition, or use according to one of claims 1-59, wherein the composition further comprises a pharmaceutically acceptable carrier, excipient, and/or diluent, optionally wherein the pharmaceutically acceptable carrier, excipient, and/or diluent is pharmaceutically acceptable for use in a human.
